(19) United States
(12) Patent Application Publication
(10) Pub. No.: US 2006/0263907 A1
(43) Pub. Date: Nov. 23, 2006

(54) Fluorescence lateral flow immunoassay

(76) Inventor: Stephen Eliot Zweig, Los Gatos, CA (US)

Correspondence Address: MORRISON & FOERSTER LLP, 12531 HIGH BLUFF DRIVE, SUITE 100, SAN DIEGO, CA, 92130-2040, US

(21) Appl. No.: 11/413,997

(22) Filed: Apr. 28, 2006

Related U.S. Application Data
(60) Provisional application No. 60/676,019, filed on Apr. 29, 2005.

(51) Int. Cl. C12M 1/34; G01N 33/551

(52) U.S. Cl. 436/524; 435/287.2; 977/900

(57) ABSTRACT

The present invention relates to devices, kits, instruments and methods for conducting lateral flow assays. A naturally hydrophilic membrane a fluorescent or luminescent label are used in the present devices, kits, instruments and methods. Preferably, a single naturally hydrophilic membrane and/or a fluorescent or luminescent particle label is used.

### CROSS-REFERENCE TO RELATED APPLICATIONS

This application claims benefit of U.S. provisional application Ser. No. 60/676,019 filed Apr. 29, 2005.

### FIELD OF THE INVENTION

The present invention relates to devices, kits, instruments and methods for conducting lateral flow assays. A naturally hydrophilic membrane and a fluorescent or luminescent label are used in the present devices, kits, instruments and methods. Preferably, a single naturally hydrophilic membrane and/or a fluorescent or luminescent particle label is used.

### BACKGROUND OF THE INVENTION

Lateral flow immunoassays are widely used in many different areas of analytical chemistry and medicine. Such assays enable relatively sophisticated chemical analysis to be quickly performed by unskilled users upon complex samples, such as urine, blood, and environmental samples, and typically return results within a few minutes using minimal amounts of additional instrumentation.

Previous lateral flow immunoassays were composed of several different types of membrane material pressed together. Typically a liquid sample will be applied to a separation membrane. This membrane separates the liquid portions of the sample from solid particles, such as red cells. The fluid portion of the sample then travels through a wicking membrane, which transports the fluid by capillary action, to a conjugate release membrane, which stores an antibody reactive with analyte present in the liquid sample. The antibody in turn is usually labeled with small light absorbing particles, such as colloidal gold particles. The antibody and colloidal gold particles will typically be stored in a dry state, and be rehydrated by the fluid sample.

As the fluid sample passes by the conjugate release membrane, the antibody and detection particles enter the fluid, and the antibody binds to a first epitope (binding region) on any analyte molecules that are present in the sample. The fluid then passes into a reaction membrane. Typically the reaction membrane will be made from a thin material, such as nitrocellulose, that is capable of binding protein. A second capture antibody, capable of binding to a different epitope on the analyte molecule, is applied to the membrane, usually in the form of a thin line that provides good visual contrast for subsequent visual assessment steps. The capture antibody binds tightly to the membrane, and remaining membrane protein binding capability is then removed by incubation with excess “blocking protein”. As the fluid passes through the reaction membrane, a final absorbent membrane that is in contact with the reaction membrane removes excess fluid. Analyte molecules in the sample bind to the capture antibody, which in turn is bound to the reaction membrane. The detection antibody in turn also binds to the reaction membrane bound analyte, and the colored detector particles bind to the detection antibody, forming a sandwich that produces a visible signal when analyte is present.

Previous lateral flow immunoassay work is exemplified by US patents and patent application publications: U.S. Pat. Nos. 5,602,040; 5,622,871; 5,656,503; 6,187,598; 6,228,660; 6,818,455; No. 2001/0008774; 2005/0244986; U.S. Pat. No. 6,352,862; No. 2003/0207465; 2003/0143755; 2003/0219908; U.S. Pat. Nos. 5,714,389; 5,989,921; 6,485,982; Ser. No. 11/035,047; U.S. Pat. Nos. 5,656,448; 5,559,041; 5,252,496; 5,728,587; 6,027,943; 6,506,612; 6,541,277; 6,737,277 B1; U.S. Pat. Nos. 5,073,484; 5,654,162; 6,020,147; 4,956,302; 5,120,643; 6,534,320; 4,942,522; 4,703,017; 4,743,560; 5,591,645; and RE 38,430 E.

Other types of diagnostic assays have also been developed that use chromatographic principles. These are exemplified by hand-held cholesterol assays, such as the work disclosed in U.S. Pat. Nos. 4,999,287; 4,987,085; 4,959,324; 5,204,063, and 5,508,664, which discuss a number of ways in which small buffer packages may be packaged in a single hand-held diagnostic unit.

Recently, Jones et. al. (“The development of a novel platform for lateral flow assays” AACC Oak Ridge conference, Apr. 29-30, 2004, Abstract 20) disclosed that a polymer treated glass fiber membrane, called “SLF5” (produced by Whatman corporation), is capable of functioning in a lateral flow immunoassay as a single membrane. Due to its unique combination of properties, SLF5 (also called “Fusion5”) combines the disparate functions of the blood separator, sample wick, conjugate release, reaction membrane, and absorbent membrane into a single glass fiber composite membrane, with fluid flow properties adequate to allow all five functions to be performed in a single structure. This membrane does not bind protein. As a result, capture antibodies are immobilized by binding them to latex beads (around 1-2 microns in diameter), which are in turn trapped by the meshwork of the membrane. Jones demonstrated that this device could function in a visual lateral flow immunoassay using visually detected gold particles.

Although traditional lateral flow immunoassay technology has been oriented towards methods that provide a directly observable visible signal, there are limitations to the visual approach. Visual approaches are difficult to quantitate, can give results that vary between users (due to the subjective nature of the visual response), and are not sensitive enough to enable the production of certain types of desirable immunoassays, such as highly sensitive whole-blood human chorionic gonadotropin (hCG) or other assays.

Low cost, hand held instruments are frequently employed in diagnostics in order to provide quantitative information, and to overcome the subjective nature of visually read assays. Such devices typically incorporate a microprocessor, display means, and optical reading means that directly interrogate the optical status of a reagent. In order to comply with modern quality control regulations, such devices also typically contain a number of failsafe sensors and algorithms to detect common user errors, and instruct the microprocessor to return error messages rather than erroneous results, when improper operating conditions are observed.

As analytical methods have advanced, it has become more common in recent years to employ fluorescence detection methodologies. Fluorescence signals tend to be more sensitive than colorimetric signals, because the noise background may be effectively eliminated by the proper choice of excitation and illumination filters.

One example of a modern fluorescence test strip-meter combination is the AvoSure prothrombin time system. This system is designed to monitor whole blood coagulation using a membrane based test strip. When whole blood is applied to the test strip, a polysulfone membrane separates the red cells from plasma, and the plasma then reacts with dry thromboplastin (a coagulation initiating chemical, which triggers the formation of a series of proteolytic enzymes in blood, eventually forming a clot) in the membrane, and a fluorescent protease substrate. When the protease substrate reacts with proteases, it becomes intensely fluorescent, and the time elapsed between the application of blood and the onset of fluorescence gives useful information as to the coagulation capability of the blood sample in question.

A small hand held meter that contains a miniature fluorescence detector reads the AvoSure test strip, and electrical resistance sensing means interact with electrodes on the Avocet test strip. As a result, the meter can detect application of sample, as well as common error conditions such as insufficient sample.

The AvoSure system is exemplified by U.S. Pat. Nos. 5,344,754; 5,418,141; 5,418,143; 5,554,531; 5,580,74; 6,061,128; D371,605; D435;020; D438,971; U.S. Pat. Nos. 6,575,900 and 6,629,057.

There is a need for improved analytical technology to overcome the lack of quantitation, subjectivity, and poor sensitivity of traditional lateral flow immunoassays. The present invention addresses this and other related needs.

### BRIEF SUMMARY OF THE INVENTION

In one aspect, the present invention provides devices for detecting an analyte in a sample.

In a first embodiment, the present invention provides a device for detecting an analyte in a sample, said device comprising a naturally hydrophilic membrane comprising a first location and a second location, wherein said first location comprises a dried first fluorescent or luminescent particle labeled binding reagent capable of binding to an analyte, if present in a sample to be tested, to form a first complex comprising said first labeled binding reagent and said analyte; said second location, downstream from said first location, comprises an immobilized second binding reagent capable of binding to said first complex, if present, to form a second complex comprising said first labeled binding reagent, said analyte and said immobilized second binding reagent; a liquid for transporting said analyte and said first labeled binding reagent to said second location to form said second complex, and whereby the presence, absence and/or amount of said analyte in said sample is determined by assessing fluorescence or luminescence comprised in said second complex at said second location.

In some examples of the above-described first embodiment of a device of the present invention, the first labeled binding reagent, the second immobilized binding reagent or both may bind specifically to the analyte. Alternatively, neither the first labeled binding reagent nor the second immobilized binding reagent binds specifically to the analyte, and a scavenger substance is used to improve detection specificity. In another example, the first labeled binding reagent, second immobilized binding reagent or both in the above device is an antibody to the analyte.

In a related embodiment, the present invention provides a method for detecting an analyte in a sample using the above device, which method comprises: a) contacting a sample with the above-described first embodiment of a device of the invention, wherein the sample is applied to a site of the membrane upstream of the second site; b) transporting an analyte, if present in said sample, and the dried first fluorescent or luminescent particle labeled binding reagent capable of binding to an analyte by a liquid to the second location to form the second complex comprising said first labeled binding reagent, said analyte and said immobilized second binding reagent at said second location; and c) determining presence, absence and/or amount of said analyte in said sample by assessing fluorescence or luminescence comprised in said second complex at said second location.

In a second embodiment, the present invention provides a device for detecting an analyte in a sample, which device comprises: a naturally hydrophilic membrane comprising a first location and a second location, wherein said first location comprises a dried fluorescent or luminescent particle labeled binding reagent capable of binding to an analyte, if present in a sample to be tested; said second location, downstream from said first location, comprises an immobilized substance capable of binding to said labeled binding reagent; a liquid is used to transport said analyte and said labeled binding reagent to said second location where said analyte and said immobilized substance compete for binding to said labeled binding reagent, and whereby the presence, absence and/or amount of said analyte in said sample is determined by assessing fluorescence or luminescence comprised in a complex comprising said immobilized substance and said labeled binding reagent at said second location.

In some examples of the above-described second embodiment of a device of the present invention, the labeled binding reagent binds specifically to the analyte. Alternatively, the labeled binding reagent does not bind specifically to the analyte, and a scavenger substance is used to improve detection specificity. In other examples, the labeled binding reagent is an antibody to the analyte. In yet other examples, the immobilized substance may comprise an analyte.

In a related embodiment, the present invention provides a method for detecting an analyte in a sample using the above device, which method comprises: a) contacting a sample with the above-described second embodiment of a device of the invention, wherein the sample is applied to a site of the membrane upstream of the second site; b) transporting an analyte, if present in said sample, and the dried first fluorescent or luminescent particle labeled binding reagent capable of binding to an analyte by a liquid to the second location, where said analyte and said immobilized substance compete for binding to said labeled binding reagent; and c) determining the presence, absence and/or amount of said analyte in said sample by assessing fluorescence or luminescence comprised in a complex comprising said labeled binding reagent and said immobilized substance at said second location.

In a third embodiment, the present invention provides a device for detecting an analyte in a sample, which device comprises: a naturally hydrophilic membrane comprising a first location and a second location, wherein said first location comprises a dried fluorescent or luminescent particle labeled substance; said second location, downstream from said first location, comprises an immobilized binding reagent capable of binding to said labeled substance and an analyte, if present in a sample to be tested; a liquid is used to transport said analyte and said labeled substance to said second location where said analyte and said labeled substance compete for binding to said immobilized binding reagent, and whereby the presence, absence and/or amount of said analyte in said sample is determined by assessing fluorescence or luminescence comprised in a complex comprising said immobilized binding reagent and said labeled substance at said second location.

In some examples of the above-described third embodiment of a device of the present invention, the immobilized binding reagent binds specifically to the analyte. Alternatively, the immobilized binding reagent does not bind specifically to the analyte, and a scavenger substance is used to improve detection specificity. In some examples, the immobilized binding reagent is an antibody to the analyte. In other examples, the labeled substance comprises an analyte.

In a related embodiment, the present invention provides a method for detecting an analyte in a sample using the above-described third embodiment of a device of the invention, which method comprises: a) contacting a sample with the above device, wherein the sample is applied to a site of the membrane upstream of the second site; b) transporting an analyte, if present in said sample, and the dried fluorescent or luminescent particle labeled substance by a liquid to the second location, where said analyte and said labeled substance compete for binding to said immobilized binding reagent; and c) determining the presence, absence and/or amount of said analyte in said sample by assessing fluorescence or luminescence comprised in a complex comprising said labeled substance and said immobilized binding reagent at said second location.

In a fourth embodiment, the present invention provides a device for detecting an analyte in a sample, which device comprises: a naturally hydrophilic membrane comprising a first location and a second location, wherein said first location comprises a dried first fluorescent or luminescent particle labeled binding reagent capable of binding to an analyte, and a dried second binding reagent capable of binding to an analyte, said second binding reagent further comprising a third binding reagent, said analyte, if present in a sample to be tested, forms a sandwich complex comprising said first labeled binding reagent, said analyte and said second binding reagent; said second location, downstream from said first location, comprises an immobilized fourth binding reagent capable of binding to said third binding reagent; a liquid is used to transport said analyte, said first labeled binding reagent and said second binding reagent to said second location whereby said sandwich complex is immobilized at said second location via binding between said third binding reagent and said immobilized fourth binding reagent, and whereby the presence, absence and/or amount of said analyte in said sample is determined by assessing fluorescence or luminescence comprised in said sandwich complex at said second location. Any suitable binding pairs may be used as the third and fourth binding reagents. For example, an antigen-antibody pair or avidin (strepavidin)-biotin pair may be used.

In a related embodiment, the present invention provides a method for detecting an analyte in a sample using the above device, which method comprises: a) contacting a sample with the above-described fourth embodiment of a device of the invention, wherein the sample is applied to a site of the membrane upstream of the second site; b) transporting an analyte, if present in said sample, and the dried first fluorescent or luminescent particle labeled binding reagent capable of binding to an analyte and the dried second binding reagent capable of binding to an analyte to the second location, where a sandwich complex comprising said first labeled binding reagent, said analyte and said second binding reagent is immobilized at said second location via binding between said third binding reagent and said immobilized fourth binding reagent; and c) determining presence, absence and/or amount of said analyte in said sample by assessing fluorescence or luminescence comprised in said sandwich complex at said second location.

In a fifth embodiment, the present invention provides a device for detecting an analyte in a sample, which device comprises: a naturally hydrophilic membrane comprising a first location and a second location, wherein said first location comprises a dried first fluorescent or luminescent particle labeled binding reagent capable of binding to an analyte, and a dried substance further comprising a second binding reagent, said analyte, if present in a sample to be tested, and said substance compete for binding to said first labeled binding reagent; said second location, downstream from said first location, comprises an immobilized third binding reagent capable of binding to said second binding reagent; a liquid is used to transport said analyte, said first labeled binding reagent and said substance to said second location whereby a complex comprising said first labeled binding reagent and said substance is immobilized at said second location via binding between said second binding reagent and said immobilized third binding reagent, and whereby the presence, absence and/or amount of said analyte in said sample is determined by assessing fluorescence or luminescence comprised in said complex comprising said first labeled binding reagent and said substance at said second location. Any suitable binding pairs may be used as the second and third binding reagents. For example, an antigen-antibody pair or avidin (strepavidin)-biotin pair may be used.

In a related embodiment, the present invention provides a method for detecting an analyte in a sample using the above device, which method comprises: a) contacting a sample with the above-described fifth embodiment of a device of the invention, wherein the sample is applied to a site of the membrane upstream of the second site; b) transporting an analyte, if present in said sample, the first fluorescent or luminescent particle labeled binding reagent capable of binding to said analyte, and the dried substance further comprising a second binding reagent to the second location, where said analyte and said substance compete for binding to said first labeled binding reagent and a complex comprising said first labeled binding reagent and said substance is immobilized at said second location via binding between said second binding reagent and said immobilized third binding reagent; and c) determining the presence, absence and/or amount of said analyte in said sample by assessing fluorescence or luminescence comprised in said complex comprising said first labeled binding reagent and said substance at said second location.

In a sixth embodiment, the present invention provides a device for detecting an analyte in a sample, which device comprises: a naturally hydrophilic membrane comprising a first location and a second location, wherein said first location comprises a dried fluorescent or luminescent particle labeled substance, and a dried first binding reagent capable of binding to an analyte, said first binding reagent further comprising a second binding reagent, said analyte, if present in a sample to be tested, and said labeled substance compete for binding to said first binding reagent; said second location, downstream from said first location, comprises an immobilized third binding reagent capable of binding to said second binding reagent; a liquid is used to transport said analyte, said labeled substance and said first binding reagent to said second location whereby a complex comprising said labeled substance and said first binding reagent is immobilized at said second location via binding between said second binding reagent and said immobilized third binding reagent, and whereby the presence, absence and/or amount of said analyte in said sample is determined by assessing fluorescence or luminescence comprised in said complex comprising said labeled substance and said first binding reagent at said second location. Any suitable binding pairs may be used as the second and third binding reagents. For example, an antigen-antibody pair or avidin (strepavidin)-biotin pair may be used.

In a related embodiment, the present invention provides a method for detecting an analyte in a sample using the above device, which method comprises: a) contacting a sample with the above-described sixth embodiment of a device of the invention, wherein the sample is applied to a site of the membrane upstream of the second site; b) transporting an analyte, if present in said sample, the dried fluorescent or luminescent particle labeled substance, and the dried first binding reagent capable of binding to said analyte, said first binding reagent further comprising a second binding reagent, to the second location, where said analyte and said labeled substance compete for binding to said first binding reagent, and a complex comprising said labeled substance and said first binding reagent is immobilized at said second location via binding between said second binding reagent and said immobilized third binding reagent; and c) determining presence, absence and/or amount of said analyte in said sample by assessing fluorescence or luminescence comprised in said complex comprising said labeled substance and said first binding reagent at said second location.

In a seventh embodiment, the present invention provides a device for detecting an analyte in a sample, which device comprises: a) a container containing a liquid or dried first fluorescent or luminescent particle labeled binding reagent capable of binding to an analyte, if present in a sample to be tested, to form a first complex comprising said first labeled binding reagent and said analyte; and b) a naturally hydrophilic membrane binding to said first complex, if present, to form a second complex comprising said first labeled binding reagent, said analyte and said immobilized second binding reagent, wherein a liquid is used to laterally transport said analyte and said first labeled binding reagent on said membrane to said test location to form said second complex, and whereby the presence, absence and/or amount of said analyte in said sample is determined by assessing fluorescence or luminescence comprised in said second complex at said test location.

In an eighth embodiment, the present invention provides a device for detecting an analyte in a sample, which device comprises: a) a container containing a liquid or dried fluorescent or luminescent particle labeled binding reagent capable of binding to an analyte, if present in a sample to be tested; and b) a naturally hydrophilic membrane comprising a test location comprising an immobilized substance capable of binding to said labeled binding reagent; wherein a liquid is used to laterally transport said analyte and said labeled binding reagent on said membrane to said test location where said analyte and said immobilized substance compete for binding to said labeled binding reagent, and whereby the presence, absence and/or amount of said analyte in said sample is determined by assessing fluorescence or luminescence comprised in a complex comprising said immobilized substance and said labeled binding reagent at said test location.

In a ninth embodiment, the present invention provides a device for detecting an analyte in a sample, which device comprises: a) a container containing a liquid or dried fluorescent or luminescent particle labeled substance; and b) a naturally hydrophilic membrane comprising a test location comprising an immobilized binding reagent capable of binding to said labeled substance and an analyte, if present in a sample to be tested; wherein a liquid is used to laterally transport said analyte and said labeled substance on said membrane to said test location where said analyte and said labeled substance compete for binding to said immobilized binding reagent, and whereby presence, absence and/or amount of said analyte in said sample is determined by assessing fluorescence or luminescence comprised in a complex comprising said immobilized binding reagent and said labeled substance at said test location.

In the above devices, the naturally hydrophilic membrane may comprise polymer treated glass fiber. For example the naturally hydrophilic membrane is SLF5 membrane. In some examples, the device may comprise a single naturally hydrophilic membrane. In other examples, the naturally hydrophilic membrane may be supported by a solid backing. In yet other examples, the membrane may extend to the opposite side of the backing.

In the above devices, the first location, second location or both locations may be in the form of a zone or zones. In some examples, the test location may be in the form of a zone or zones.

In the above devices, the fluorescent particle may comprise a quantum dot. In other examples, the fluorescent particle may be a transfluorosphere or latex microsphere bead. In some examples, the fluorescent or luminescent particle labeled binding reagent or substance is air dried or lyophilized. The fluorescent or luminescent particle labeled binding reagent or substance may be dried in the presence of a material that: a) stabilizes the fluorescent or luminescent particle labeled binding reagent or substance; b) facilitates resuspension of the fluorescent or luminescent particle labeled binding reagent or substance in the liquid; and/or c) facilitates mobility of the fluorescent or luminescent particle labeled binding reagent or substance. In some examples, the fluorescent or luminescent particle labeled binding reagent or substance is a protein, a peptide, a polysaccharide, a sugar, a polymer, a gelatin or a detergent.

In the above devices, the second binding reagent may be immobilized at the second location by absorption, adsorption, or covalent binding to the naturally hydrophilic membrane; or attached to another substance or particle that is immobilized to the naturally hydrophilic membrane.

In the above devices, a sample liquid alone may be used to transport the analyte and the labeled binding reagent to the second location. Alternatively, a developing liquid may be used to transport the analyte and the labeled binding reagent to the second location.

The above devices may further comprise a housing. In some examples, the housing covers at least the first and second locations on the membrane, and comprises a sample application site to allow sample application upstream from or to the first location on the membrane and an opening around the second location to allow fluorescence or luminescence detection at the second location. The housing may comprise a plastic material.

The above devices may further comprise a liquid holder and a means for transporting a liquid in the liquid holder to the first location or upstream from the first location on the membrane. For example, the liquid may be transported from the liquid holder to the membrane by a relative position change between the liquid holder and the membrane.

The above devices may further comprise a pair of electrodes on the membrane, wherein the pair of electrodes are spatially separated at the sample application site and at a site downstream from the second location on the membrane such that sample application and the liquid flow to the second location may be electrically monitored.

The above devices may further comprise a sample receiving member upstream from and in fluid communication with the first location, wherein sample receiving member is supported by the solid backing, and the sample is applied to the sample receiving member and then transported to the first and second locations sequentially. In some examples, the devices may further comprise a housing that covers the first and second locations on the membrane and at least a portion of the sample receiving member, wherein the housing comprises an opening around the second location to allow fluorescence or luminescence detection at the second location. In other examples, at least a portion of the sample receiving member is not covered by the housing and a sample is applied to the sample receiving member portion outside the housing and then transported to the first and second locations sequentially. In yet other examples, the first and second locations on the membrane and the sample receiving member are entirely covered by the housing, and the housing comprises a sample application site to allow sample application to the sample receiving member.

In the above devices, the second location on the membrane may comprise an additional immobilized binding reagent capable of binding to a second fluorescent or luminescent label, wherein the binding between the additional binding reagent and the second fluorescent or luminescent label is independent of the binding between the analyte in the sample and its respective immobilized binding reagent at the second location, and the two fluorescent or luminescent signals at the second location may be detected at two distinct spectra without interference from each other.

The above devices may comprise two different fluorescent or luminescent particle labeled binding reagents capable of binding to a first epitope of an analyte at the first location, wherein one labeled binding reagent has low binding affinity to the analyte and the other labeled binding reagent has high binding affinity to the analyte;

two different immobilized binding reagents capable of binding to a second epitope of the analyte, wherein one immobilized binding reagent has low binding affinity to the analyte and the other immobilized binding reagent has high binding affinity to the analyte;

at low analyte concentration, a sandwich of the analyte with the two low affinity antibodies is formed and at high analyte concentration, a sandwich of the analyte with the two high affinity antibodies is formed; and

two different fluorescent or luminescent particle labels may be detected at the same or different spectra.

The above devices may further comprise a control location downstream from, but in fluid communication with, the second location, wherein the control location comprises means for indicating a valid test result.

The above devices may further comprise a liquid absorption pad downstream from, but in fluid communication with, the second location.

In yet another embodiment, the present invention provides a lateral flow device for detection of an analyte in a sample fluid; wherein said analyte comprises at least two distinct epitopes, said lateral flow device comprising: a single membrane; mobile fluorescent or luminescent detection moieties, present on a storage zone of said membrane, capable of binding to a first epitope on said analyte; capture moieties, capable of binding to a second epitope on said analyte, immobilized on a detection zone in said membrane; a sample application zone on said membrane; and a user controlled reservoir containing a transport liquid; wherein after application of the sample, user controlled manipulation of the reservoir releases a transport liquid onto the membrane, transporting the analyte to the fluorescent or luminescent detection moieties, which bind to a first epitope on the analyte, and then transports the analyte and mobile fluorescent or luminescent detection moieties to the detection zone on said membrane, where capture moieties bind to a second epitope on said analyte, producing a detectable signal. Any suitable detection moieties may be used. For example the detection moieties may be antibodies, receptors or lectins.

In yet another embodiment, the present invention provides a lateral flow device for detection of an analyte in a sample fluid; wherein said analyte comprises at least two distinct epitopes, said lateral flow device comprising: mobile detection moieties present on a storage zone on a membrane, preferably a single membrane, which generate a detectable signal, capable of binding to a first epitope on said analyte; capture moieties, capable of binding to a second epitope on said analyte, immobilized on a detection zone in said membrane; a sample application zone on said membrane; and sample detection electrodes associated with said sample application zone on said membrane; wherein application of sample to said application zone produces a change in an electrical property across said sample detection electrodes, enabling the time of sample application to be automatically assessed by an instrument that monitors the electrical state of said sample detection electrodes.

In yet another embodiment, the present invention provides a lateral flow device for detection of an analyte in a sample fluid; wherein said analyte comprises at least two distinct epitopes, said lateral flow device comprising: mobile detection moieties present on a storage zone on said membrane, which generate a detectable signal, and are capable of binding to a first epitope on said analyte; capture moieties, capable of binding to a second epitope on said analyte, immobilized on a detection zone in said membrane; a sample application zone on said membrane; and fluid transport detection electrodes associated with said detection zone on said membrane; wherein fluid transport occurring after application of sample to said application zone produces a change in an electrical property across said detection electrodes, enabling the time of sample transport past the detection zone region to be automatically assessed by an instrument that monitors the electrical state of said detection zone electrodes.

In another aspect, the present invention provides an instrument for reading a lateral flow immunoassay device, said instrument comprising: a fluorescence or luminescence detector capable of reading a detection zone on said immunoassay device; electrodes capable of interfacing with electrodes on said lateral flow immunoassay device; and computation means, wherein travel of fluid in the lateral flow immunoassay device causes a change in electrical property across the electrodes on said lateral flow immunoassay device, said change in electrical property is communicated to the instrument via the instrument's interface electrodes, and the information is transmitted to the instrument's computational means.

In still another aspect, the present invention provides a validation method for a lateral flow device for detection of an analyte in a sample fluid, wherein said validation method comprises; an analyte detection particle and a control detection particle said analyte detection particle containing means to specifically bind to a first epitope on the analyte; said analyte detection particle emitting a first detectable signal; said control detection particle containing means to specifically bind to a region on a control non-analyte molecule; said control detection particle emitting a second detectable signal, which may be distinguished from the first detectable signal; a detection zone on said lateral flow device containing means to specifically bind to a second epitope on the analyte, said means being immobilized to avoid migration away from said detection zone; said detection zone further containing means to specifically bind to a region on said control non-analyte molecule that is different from the region bound by the control detection particle; said means being immobilized to avoid migration away from said detection zone; said analyte binding means and said control binding means being intermixed in the same region of said detection zone; wherein validation of said lateral flow device is achieved by monitoring the binding or non binding of the control particle to the control non-analyte molecule by means of the second detectable signal emitted by the control particle.

In yet another aspect, the present invention provides a method for extending the analyte detection dynamic range of a lateral flow device for detection of an analyte in a sample fluid, comprising: a high affinity analyte detection particle and a high affinity detection particle, said high affinity analyte detection particle containing means to specifically bind to a first epitope on the analyte with a strong binding force; said high affinity analyte detection particle emitting a first detectable signal; and a low affinity analyte detection particle; said low affinity analyte detection particle containing means to specifically bind to an epitope on the analyte with a weak binding force; said low affinity detection particle emitting a second detectable signal, which may be distinguished from the first detectable signal; a detection zone on said lateral flow device containing means to specifically bind to a second epitope on the analyte, said means being immobilized to avoid migration away from said detection zone; wherein low analyte concentrations are detected by monitoring the binding or non binding of the high affinity detection particle to the detection zone by means of the first detectable signal, and high analyte concentrations are detected by monitoring the binding or non-binding of the low affinity detection particle to the detection zone by means of the second detectable signal.

In yet another aspect, the present invention provides a method to modify the surface properties of a particle to render the particle, when suspended in an aqueous carrier solvent, capable of migrating within the matrix of a bibulous membrane without adverse interaction with other particles or with the bibulous membrane matrix, said method comprising: covalently modifying the surface of the particle with one or more ligands, said ligands being capable of binding to hydrophilic molecules by a covalent or non-covalent bond; said hydrophilic molecules being soluble in said aqueous carrier solvent; wherein said hydrophilic molecules protect the particle from adverse interactions with other particles or with the bibulous membrane matrix. The adverse interaction may comprise particle aggregation or particle sticking to the bibulous membrane matrix. Examples of ligands which may be used to practice this aspect of the invention include but are not limited to strepavidin or avidin, and said hydrophilic molecule may be biotinated bovine serum albumin, or other biotinated hydrophilic protein, and the binding reaction is quenched with excess biotin. The ligands may be hydrophilic, and may be capable of protecting the particle from adverse interactions with other particles or with the bibulous membrane matrix without the need of binding to additional hydrophilic molecules.

The devices and methods of the invention may be used by applying sample to a first location of the membrane, or the sample may be applied to a site of the membrane upstream of the first location of the membrane. In some examples, a sample liquid alone is used to transport the analyte and the labeled binding reagent to the second location. Alternatively, a developing liquid is used to transport the analyte and the labeled binding reagent to the second location.

The devices and methods of the invention may be used to detect analyte from a biological sample, including but not limited to whole blood, a serum, a plasma or a urine sample. In some examples, the analyte may be a cell, a virus or a molecule. Examples of analyte that may be detected using the devices and methods of the invention include but are not limited to hCG, hLH, hFSH, hTSH, an antigen of an infectious organism, an antibody to an infectious organism, or a disease marker.

### BRIEF DESCRIPTION OF THE DRAWINGS

FIGS. 1A-1C show an overview of an exemplary lateral flow device interacting with a meter optics block and an electrical interface.

FIGS. 2A-2C show exemplary electrical sensors used to monitor sample application and fluid flow.

FIGS. 3A-3B show the fluid reservoir and fluid flow after sample application to an exemplary lateral flow device.

FIG. 4 show a diagram of resistance versus time as reported by the device's fluid sensors shown in FIG. 2.

FIG. 5 shows a diagram of fluorescence versus time in two cases where analyte is, present or absent.

FIG. 6 shows the use two separate detector particles, in which one particle detects analyte, and the other particle serves to generate a control signal used to determine the validity of the analyte signal.

FIG. 7 shows the use of two separate detector particles, in which one particle is used to determine low levels of analyte, and the other particle is used to detect high levels of analyte.

FIG. 8 illustrates a method by which migration of particles within the matrix of a porous bibulous membrane may be enhanced.

FIGS. 9A-9B show the resistance and fluorescent data obtained from one embodiment of a lateral flow device of the invention.

FIGS. 10A-10B show a dose response curve of an exemplary hCG test.

### DETAILED DESCRIPTION OF THE INVENTION

### A. Definitions

Unless defined otherwise, all technical and scientific terms used herein have the same meaning as is commonly understood by one of ordinary skill in the art to which this invention belongs. All patents, patent applications (published or unpublished), and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

As used herein, “antibody” is used in the broadest sense. Therefore, an “antibody” may be naturally occurring or man-made such as monoclonal antibodies produced by conventional hybridoma technology and/or a functional fragment thereof. Antibodies of the present invention comprise monoclonal and polyclonal antibodies as well as fragments (such as Fab, Fab′, F(ab′)₂, Fv) containing the antigen-binding domain and/or one or more complementarity determining regions of these antibodies.

As used herein, “monoclonal antibody” refers to an antibody obtained from a population of substantially homogeneous antibodies, i.e., the antibodies comprising the population are identical except for possible naturally occurring mutations that are present in minor amounts. As used herein, a “monoclonal antibody” further refers to functional fragments of monoclonal antibodies.

As used herein, “mammal” refers to any of the mammalian class of species, preferably human (including humans, human subjects, or human patients). Mammals include, but are not limited to, farm animals, sport animals, pets, primates, horses, dogs, cats, mice and rats.

As used herein, “treatment” means any manner in which the symptoms of a condition, disorder or disease are ameliorated or otherwise beneficially altered. Treatment also encompasses any pharmaceutical use of the compositions herein.

As used herein, “disease or disorder” refers to a pathological condition in an organism resulting from, e.g., infection or genetic defect, and characterized by identifiable symptoms.

As used herein, the term “subject” is not limited to a specific species or sample type. For example, the term “subject” may refer to a patient, and frequently a human patient. However, this term is not limited to humans and thus encompasses a variety of mammalian species.

As used herein, “afflicted” as it relates to a disease or disorder refers to a subject having or directly affected by the designated disease or disorder.

As used herein the term “sample” refers to anything which may contain an analyte for which an analyte assay is desired. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregate of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s).

As used herein, the term “specifically binds” refers to the specificity of an antibody such that it preferentially binds to a defined target. An antibody “specifically binds” to a target if it binds with greater affinity, avidity, more readily, and/or with greater duration than it binds to other substances. For example, an antibody that specifically binds to a target may bind to the target with at least about 10%, at least about 20%, at least about 30%, at least about 50%, at least about 60%, at least about 70%, at least about 80%, at least about 90% or more, greater affinity as compared to binding to other substances; or with at least about two-fold, at least about five-fold, at least about ten-fold or more of the affinity for binding to other substances. Recognition by an antibody of a particular target in the presence of other potential targets is one characteristic of such binding. In one embodiment, the specific binding reagent can distinguish the analyte from other substances that are present or likely to be present in the sample to be tested.

### B. Devices and Methods for Detecting an Analyte in a Sample

In one aspect, the present invention provides devices for detecting an analyte in a sample. In one embodiment, the present invention provides a device for detecting an analyte in a sample, which device comprises: a naturally hydrophilic membrane comprising two separate first and second locations, wherein said first location comprises a dried first fluorescent or luminescent particle labeled binding reagent capable of binding to an analyte, if present in a sample to be tested, to form a first complex comprising said first labeled binding reagent and said analyte; said second location, downstream from said first location, comprises an immobilized second binding reagent capable of binding to said first complex, if present, to form a second complex comprising said first labeled binding reagent, said analyte and said immobilized second binding reagent; a liquid is used to transport said analyte and said first labeled binding reagent to said second location to form said second complex, and whereby presence, absence and/or amount of said analyte in said sample is determined by assessing fluorescence or luminescence comprised in said second complex at said second location.

In a related embodiment, the present invention provides a method for detecting an analyte in a sample using the above device, which method comprises: a) contacting a sample with the above device, wherein the sample is applied to a site of the membrane upstream of the second site; b) transporting an analyte, if present in said sample, and the dried first fluorescent or luminescent particle labeled binding reagent capable of binding to an analyte by a liquid to the second location to form the second complex comprising said first labeled binding reagent, said analyte and said immobilized second binding reagent at said second location; and c) determining presence, absence and/or amount of said analyte in said sample by assessing fluorescence or luminescence comprised in said second complex at said second location.

In another embodiment, the present invention provides a device for detecting an analyte in a sample, which device comprises: a naturally hydrophilic membrane comprising two separate first and second locations, wherein said first location comprises a dried fluorescent or luminescent particle labeled binding reagent capable of binding to an analyte, if present in a sample to be tested; said second location, downstream from said first location, comprises an immobilized substance capable of binding to said labeled binding reagent; a liquid is used to transport said analyte and said labeled binding reagent to said second location where said analyte and said immobilized substance compete for binding to said labeled binding reagent, and whereby presence, absence and/or amount of said analyte in said sample is determined by assessing fluorescence or luminescence comprised in a complex comprising said immobilized substance and said labeled binding reagent at said second location.

In a related embodiment, the present invention provides a method for detecting an analyte in a sample using the above device, which method comprises: a) contacting a sample with the above device, wherein the sample is applied to a site of the membrane upstream of the second site; b) transporting an analyte, if present in said sample, and the dried first fluorescent or luminescent particle labeled binding reagent capable of binding to an analyte by a liquid to the second location, where said analyte and said immobilized substance compete for binding to said labeled binding reagent; and c) determining presence, absence and/or amount of said analyte in said sample by assessing fluorescence or luminescence comprised in a complex comprising said labeled binding reagent and said immobilized substance at said second location.

In still another embodiment, the present invention provides a device for detecting an analyte in a sample, which device comprises: a naturally hydrophilic membrane comprising two separate first and second locations, wherein said first location comprises a dried fluorescent or luminescent particle labeled substance; said second location, downstream from said first location, comprises an immobilized binding reagent capable of binding to said labeled substance and an analyte, if present in a sample to be tested; a liquid is used to transport said analyte and said labeled substance to said second location where said analyte and said labeled substance compete for binding to said immobilized binding reagent, and whereby presence, absence and/or amount of said analyte in said sample is determined by assessing fluorescence or luminescence comprised in a complex comprising said immobilized binding reagent and said labeled substance at said second location.

In a related embodiment, the present invention provides a method for detecting an analyte in a sample using the above device, which method comprises: a) contacting a sample with the above device, wherein the sample is applied to a site of the membrane upstream of the second site; b) transporting an analyte, if present in said sample, and the dried fluorescent or luminescent particle labeled substance by a liquid to the second location, where said analyte and said labeled substance compete for binding to said immobilized binding reagent; and c) determining presence, absence and/or amount of said analyte in said sample by assessing fluorescence or luminescence comprised in a complex comprising said labeled substance and said immobilized binding reagent at said second location.

Any suitable naturally hydrophilic membrane may be used. For example, a naturally hydrophilic membrane comprising polymer treated glass fiber may be used. Preferably, a SLF5 membrane (Jones et al., “The development of a novel platform for lateral flow assays,” Oak Ridge Conference, Pushing the Technology Envelope: An Exploration of the Future of Clinical laboratory Testing, Apr. 29-30, 2004—San Jose, Calif.) or Whatman FUSION 5™ membrane is used.

The devices may comprise any suitable numbers of the naturally hydrophilic membrane. In one example, the devices comprise a single naturally hydrophilic membrane. In another example, the devices comprise multiple naturally hydrophilic membranes, whether the same type of different types of the naturally hydrophilic membranes.

The various locations on the membrane, including the first, second and/or the control locations, can take any suitable forms. The locations may be dots, circles, squares, zones or lines, etc. In one example, the first, second and/or the control locations are in the form of a zone or zones, or a line or lines. Preferably, the zone(s) or line(s) extends across the width of the membrane.

Any suitable fluorescent or luminescent label may be used. Preferably, a fluorescent or luminescent particle label is used. The exemplary fluorescent particle labels include a quantum dot, a transfluorosphere (e.g., bead) and a fluorescent microsphere (e.g., a green latex microsphere bead). Preferably, the fluorescent particle comprises a quantum dot.

The fluorescent or luminescent labeled binding reagent or substance, e.g., a quantum dot or a transfluorosphere or fluorescent microsphere bead, may be dried on the membrane or in a container, e.g., a test tube, by any suitable method, provided that the dried labeled binding reagent or substance may be transported by a liquid, e.g., a sample liquid and/or a developing liquid, to the second or test location. The dried labeled binding reagent or substance may be air dried or lyophilized. In some embodiments, the fluorescent or luminescent particle labeled binding reagent or substance is dried in the presence of a material that: a) stabilizes the fluorescent or luminescent particle labeled binding reagent or substance; b) facilitates resuspension of the fluorescent or luminescent particle labeled binding reagent or substance in the liquid; and/or c) facilitates mobility of the fluorescent or luminescent particle labeled binding reagent or substance. Any suitable material may be used for stabilizing, or facilitating resuspension and/or the mobility of the labeled reagent or substance. Exemplary materials include a protein, a peptide, a polysaccharide, a sugar, a polymer, a gelatin and a detergent.

Any suitable combination of binding reagents or substances with desired binding specificity to the analyte or analyte analog may be used to achieve the desired assay specificity. In one example, a sandwich assay format is used, and one or both of the first labeled binding reagent and the second immobilized binding reagent binds specifically to the analyte. In another example, a sandwich assay format is used, and neither the first labeled binding reagent nor the second immobilized binding reagent binds specifically to the analyte, and a scavenger substance is used to improve detection specificity. For example, if the analyte is hCG, one or both binding reagents, e.g., antibodies, can bind to different epitopes of hCG specifically. Alternatively, neither binding reagents or antibodies can bind to hCG specifically, and an anti-LH scavenger antibody is used to achieve the desired specificity for the hCG assay. Any suitable binding reagents may be used. For example, one or both of the first labeled binding reagent and the second immobilized binding reagent are antibodies to the analyte.

In another example, a competition assay format is used in which the analyte in the sample and an immobilized analyte or analyte analog compete for binding to a labeled binding reagent. In this format, the labeled binding reagent can bind specifically to the analyte. Alternatively, the labeled binding reagent does not bind specifically to the analyte, and a scavenger substance is used to achieve desired assay specificity. Any suitable binding reagents may be used. For example, the labeled binding reagent may be an antibody to the analyte. Any suitable immobilized substance may be used. For example, the immobilized substance comprises an analyte or analyte analog.

In still another example, a competition assay format is used in which the analyte in the sample and a labeled analyte or analyte analog compete for binding to an immobilized binding reagent capable of binding to both the analyte or labeled analyte or analyte analog. In this format, the immobilized binding reagent binds specifically to the analyte. Alternatively, the immobilized binding reagent does not bind specifically to the analyte, and a scavenger substance is used to achieve the desired assay specificity. Any suitable binding reagents may be used. For example, the immobilized binding reagent may be an antibody to the analyte. Any suitable labeled substance may be used. For example, the labeled substance can comprise an analyte.

The binding reagent or the substance may be immobilized at the second or test location or the control location by any suitable methods. For example, binding reagent or the substance may be immobilized by absorption, adsorption, or covalent binding to the naturally hydrophilic membrane, or by attaching to another substance or particle that is immobilized to the desired location.

Both sample liquid and other liquid may be used to transport the analyte and the labeled binding reagent or substance to the second or test location, or to control or beyond the control location, or to the distal end of the membrane, or to an absorbent pad downstream, but in fluid communication with the membrane. In one example, a sample liquid alone is used to transport the analyte and the labeled binding reagent or substance to the desired location. This is often used when sufficient sample volume, e.g., a urine sample, is available. In another example, a developing liquid is used to transport the analyte and the labeled binding reagent or substance to the desired location. A developing liquid is often used to transport an analyte in a non-liquid, e.g., solid, sample. A developing liquid is often used when a liquid sample, blood sample, is tested but the sample volume itself is not sufficient to transport the analyte and other reagents or substances to the desired location.

The device can comprise a suitable support, e.g., supported by a solid backing. Any suitable solid backing may be used. In some embodiments, the membrane can extend to the opposite side of the backing to increase the liquid flow path.

The device, whether having a backing or not, can comprise a housing that covers at least the first and second locations on the membrane, wherein the housing comprises a sample application port to allow sample application upstream from or to the first location on the membrane and an optic opening around the second location to allow fluorescence or luminescence detection at the second location. The housing may be made of any suitable material. For example, the housing comprises a plastic material.

When a developing liquid is used, the developing liquid may be placed in a holder separate from the device, e.g., a test tube. Alternatively, the device can further comprise a liquid holder and a means for transporting a liquid in the liquid holder to the first location or upstream from the first location on the membrane. The liquid may be transported from the liquid holder to the membrane by any suitable means. In one example, the liquid is transported from the liquid holder to the membrane by a relative position change between the liquid holder and the membrane.

The device can further comprise a pair of electrodes on the membrane, wherein the pair of electrodes are spatially separated at the sample application site and at a site just down stream from the second location on the membrane such that sample application and the liquid's pass of the second location may be electrically monitored.

In one embodiment, the device can further comprise, at the second or test location on the membrane, an additional immobilized binding reagent capable of binding to a second fluorescent or luminescent label, wherein the binding between the additional binding reagent and the second fluorescent or luminescent label is independent of the binding or competition between the analyte in the sample and its respective immobilized binding reagent or competing substance at the second location, and the two fluorescent or luminescent signals at the second location may be detected at two distinct spectra without interference from each other. In this design, the second location can serve both as the test location and a control location.

In another embodiment, the device can comprise two different fluorescent or luminescent particle labeled binding reagents capable of binding to a first epitope of an analyte at the first location, wherein one labeled binding reagent has low binding affinity to the analyte and the other labeled binding reagent has high binding affinity to the analyte; two different immobilized binding reagents capable of binding to a second epitope of the analyte, wherein one immobilized binding reagent has low binding affinity to the analyte and the other immobilized binding reagent has high binding affinity to the analyte; at low analyte concentration, a sandwich of the analyte with the two low affinity antibodies is formed and at high analyte concentration, a sandwich of the analyte with the two high affinity antibodies is formed; and two different fluorescent or luminescent particle labels may be detected at the same or different spectra. In this design, the detection range is extended.

The device can further comprise a control location downstream from, but in fluid communication with, the second location, wherein the control location comprises means for indicating a valid test result.

The device can further comprise a liquid absorption pad downstream from, but in fluid communication with, the second location.

The device can further comprise a sample receiving member upstream from, but in fluid communication with, the first location, wherein sample receiving member is supported by the solid backing, and the sample is applied to the sample receiving member and then transported to the first and second locations sequentially.

The device can further comprise a housing that covers the first and second locations on the membrane and at least a portion of the sample receiving member, wherein the housing comprises an optic opening around the second location to allow fluorescence or luminescence detection at the second location. In one example, at least a portion of the sample receiving member is not covered by the housing and a sample is applied to the sample receiving member portion outside the housing and then transported to the first and second locations sequentially. In another example, the first and second locations on the membrane and the sample receiving member are entirely covered by the housing and the housing comprises a sample application port to allow sample application to the sample receiving member.

In carrying out the assay, the sample may be applied to any suitable location upstream of the second or test location. In one example, the sample is applied to the first location of the membrane. In another example, the sample is applied to a site of the membrane upstream of the first location of the membrane.

The present devices and methods may be used to detect an analyte in any suitable sample. The exemplary samples include a whole blood, a serum, a plasma and a urine sample.

The present devices and methods may be used to detect any suitable analyte. For example, an analyte may be a cell, a virus and a molecule.

Non-limiting examples of cells include animal cells, plant cells, fungi, bacteria, recombinant cells or cultured cells. Animal, plant cells, fungus, bacterium cells to be detected may be derived from any genus or subgenus of the Animalia, Plantae, fungus or bacterium kingdom. Cells derived from any genus or subgenus of ciliates, cellular slime molds, flagellates and microsporidia can also be detected. Cells derived from birds such as chickens, vertebrates such fish and mammals such as mice, rats, rabbits, cats, dogs, pigs, cows, ox, sheep, goats, horses, monkeys and other non-human primates, and humans may be detected by the present devices and methods.

For animal cells, cells derived from a particular tissue to organ may be detected. For example, connective, epithelium, muscle or nerve tissue cells may be detected. Similarly, cells derived from an accessory organ of the eye, annulospiral organ, auditory organ, Chievitz organ, circumventricular organ, Corti organ, critical organ, enamel organ, end organ, external female genital organ, external male genital organ, floating organ, flower-spray organ of Ruffini, genital organ, Golgi tendon organ, gustatory organ, organ of hearing, internal female genital organ, internal male genital organ, intromittent organ, Jacobson organ, neurohemal organ, neurotendinous organ, olfactory organ, otolithic organ, ptotic organ, organ of Rosenmüller, sense organ, organ of smell, spiral organ, subcommissural organ, subformical organ, supernumerary organ, tactile organ, target organ, organ of taste, organ of touch, urinary organ, vascular organ of lamina terminalis, vestibular organ, vestibulocochlear organ, vestigial organ, organ of vision, visual organ, vomeronasal organ, wandering organ, Weber organ and organ of Zuckerkandl may be detected. Preferably, cells derived from an internal animal organ such as brain, lung, liver, spleen, bone marrow, thymus, heart, lymph, blood, bone, cartilage, pancreas, kidney, gall bladder, stomach, intestine, testis, ovary, uterus, rectum, nervous system, gland, internal blood vessels, etc may be detected. Further, cells derived from any plants, fungi such as yeasts, bacteria such as eubacteria or archaebacteria may be detected. Recombinant cells derived from any eukaryotic or prokaryotic sources such as animal, plant, fungus or bacterium cells can also be detected. Cells from various types of body fluid such as blood, urine, saliva, bone marrow, sperm or other ascitic fluids, and subfractions thereof, e.g., serum or plasma, can also be detected.

Molecules may be inorganic molecules such as ions, organic molecules or a complex thereof. Non-limiting examples of ions include sodium, potassium, magnesium, calcium, chlorine, iron, copper, zinc, manganese, cobalt, iodine, molybdenum, vanadium, nickel, chromium, fluorine, silicon, tin, boron or arsenic ions. Non-limiting examples of organic molecules include amino acids, peptides, proteins, nucleosides, nucleotides, oligonucleotides, nucleic acids, vitamins, monosaccharides, oligosaccharides, carbohydrates, lipids or a complex thereof.

Any amino acids may be detected by the present devices and methods. For example, a D- and a L-amino-acid may be manipulated. In addition, any building blocks of naturally occurring peptides and proteins including Ala (A), Arg (R), Asn (N), Asp (D), Cys (C), Gln (O), Glu (E), Gly (G), His (H), Ile (I), Leu (L), Lys (K), Met (M), Phe (F), Pro (P) Ser (S), Thr (T), Trp (W), Tyr (Y) and Val (V) may be detected.

Any proteins or peptides may be detected by the present devices and methods. For example, membrane proteins such as receptor proteins on cell membranes, enzymes, transport proteins such as ion channels and pumps, nutrient or storage proteins, contractile or motile proteins such as actins and myosins, structural proteins, defense protein or regulatory proteins such as antibodies, hormones and growth factors may be detected. Proteineous or peptidic antigens can also be detected.

Any nucleic acids, including single-, double and triple-stranded nucleic acids, may be detected by the present devices and methods. Examples of such nucleic acids include DNA, such as A-, B- or Z-form DNA, and RNA such as mRNA, tRNA and rRNA.

Any nucleosides may be detected by the present devices and methods. Examples of such nucleosides include adenosine, guanosine, cytidine, thymidine and uridine. Any nucleotides may be detected by the present devices and methods. Examples of such nucleotides include AMP, GMP, CMP, UMP, ADP, GDP, CDP, UDP, ATP, GTP, CTP, UTP, dAMP, dGMP, dCMP, dTMP, dADP, dGDP, dCDP, dTDP, dATP, dGTP, dCTP and dTTP.

Any vitamins may be detected by the present devices and methods. For example, water-soluble vitamins such as thiamine, riboflavin, nicotinic acid, pantothenic acid, pyridoxine, biotin, folate, vitamin B₁₂ and ascorbic acid may be detected. Similarly, fat-soluble vitamins such as vitamin A, vitamin D, vitamin E, and vitamin K may be detected.

Any monosaccharides, whether D- or L-monosaccharides and whether aldoses or ketoses, may be detected by the present devices and methods. Examples of monosaccharides include triose such as glyceraldehyde, tetroses such as erythrose and threose, pentoses such as ribose, arabinose, xylose, lyxose and ribulose, hexoses such as allose, altrose, glucose, mannose, gulose, idose, galactose, talose and fructose and heptose such as sedoheptulose.

Any lipids may be detected by the present devices and methods. Examples of lipids include triacylglycerols such as tristearin, tripalmitin and triolein, waxes, phosphoglycerides such as phosphatidylethanolamine, phosphatidylcholine, phosphatidylserine, phosphatidylinositol and cardiolipin, sphingolipids such as sphingomyelin, cerebrosides and gangliosides, sterols such as cholesterol and stigmasterol and sterol fatty acid esters. The fatty acids may be saturated fatty acids such as lauric acid, myristic acid, palmitic acid, stearic acid, arachidic acid and lignoceric acid, or may be unsaturated fatty acids such as palmitoleic acid, oleic acid, linoleic acid, linolenic acid and arachidonic acid.

In one example, the analyte to be detected is hCG, hLH, hFSH, hTSH, an antigen of an infectious organism, an antibody to an infectious organism and disease marker.

### C. Methods to Modify the Surface Properties of a Particle

In yet another aspect, the present invention provides a method to modify the surface properties of a particle to render the particle, when suspended in an aqueous carrier solvent, capable of migrating within the matrix of a bibulous membrane without adverse interaction with other particles or with the bibulous membrane matrix, said method comprising: covalently modifying the surface of the particle with one or more ligands, said ligands being capable of binding to hydrophilic molecules by a covalent or tight non-covalent bond; said hydrophilic molecules being soluble in said aqueous carrier solvent; wherein said hydrophilic molecules protect the particle from adverse interactions with other particles or with the bibulous membrane matrix.

The present method may be used to reduce any adverse interaction among particles or between particles and the bibulous membrane matrix. The adverse interaction that may be reduced include particle aggregation or particle sticking to the bibulous membrane matrix.

Any suitable ligand and hydrophilic molecule may be used. In one example, the ligand is strepavidin or avidin, and the hydrophilic molecule is biotinated bovine serum albumin, or other biotinated hydrophilic protein, and the binding reaction is quenched with excess biotin. In another example, the ligands are hydrophilic, and are capable of protecting the particle from adverse interactions with other particles or with the bibulous membrane matrix without the need of binding to additional hydrophilic molecules.

### D. Exemplary Embodiments

This disclosure discusses an improved lateral flow immunoassay that is designed for low cost production, high sensitivity, reliability, broad dynamic range, and compatibility with low-cost, hand-held fluorescence readers.

The example is a disposable lateral flow immunoassay device that uses a single membrane for all fluid pathways, and fluorescent particles to achieve higher detection efficiency. For high reliability operation, timing of sample application and fluid flow through the device is monitored by electrical means.

In another aspect of the example, multiple fluorescent particles emitting a fluorescent or luminescent signal at two distinct wavelengths are disclosed. Here, the two particles bind to the same detection zone on the lateral flow immunoassay, and are detected by a single optical fluorescence detector that is capable of distinguishing the two distinct wavelengths. Use of such dual particle methods can lead to improved lateral flow immunoassays with improved internal validation (control) capability, and/or broader dynamic range.

In a first improvement, a fluorescent particle, reactive to the test analyte, binds to an analyte that is held in a detection zone by immobilized capture antibodies against the analyte, emitting a first fluorescent signal. This first test signal is validated by a control signal using control fluorescent particles reactive to control material that is bound to the same detection region as the test analyte. The binding of control particles produces a second fluorescent signal indicative of the fitness for use of the first (test) fluorescent signal.

In a second improvement, a fluorescent particle with a high affinity to the test analyte efficiently detects low levels of analyte that are bound to the detection zone by immobilized high affinity capture antibodies against the analyte, emitting a first fluorescence signal that covers the high sensitivity region of the assay. A second fluorescent particle with a low affinity to the test analyte detects higher levels of analyte bound to the detection zone by immobilized low affinity capture antibodies, emitting a second fluorescence signal that covers the low analyte sensitivity region of the assay. This dual, low range, high range system extends the dynamic range of the assay, enabling the assay to function over both low and high analyte range, while minimizing distortion due to excess amounts of target analyte saturating the binding capability of the particle and capture antibodies.

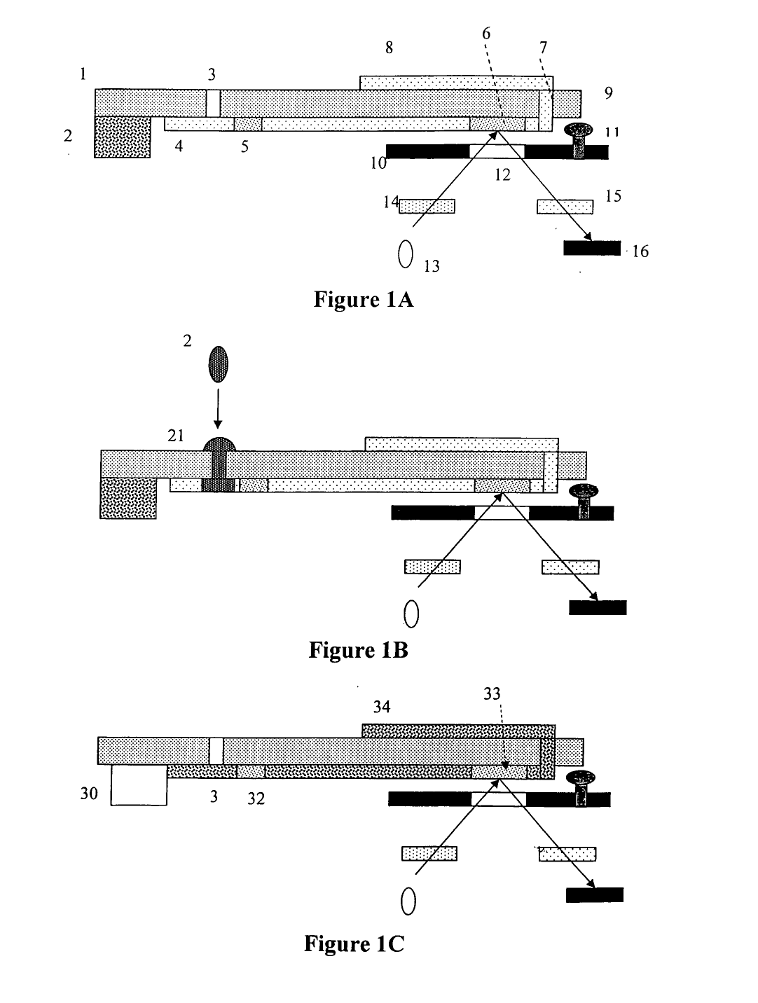

FIGS. 1A-1C show one embodiment of a device of the present invention. The device contains a plastic support (1), and a chamber containing a running buffer (2) attached to plastic support (1). Plastic support (1) also contains a sample port (3). Underneath sample port (3) is a lateral flow membrane (4), such as Whatman Fusion 5 membrane, or the like. Lateral flow membrane (4) contains a region (5) where a detection conjugate, typically an antibody to the test analyte conjugated to a fluorescent particle, is stored in a dry form. Membrane (4) additionally contains a detection region (6) containing a capture antibody, such as a second analyte against a different epitope on the analyte than the detection antibody. Often region (6), as shown in FIG. 1A, will contain such capture antibody bound to microbeads that are too large to migrate in the membrane, and thus remain stationary.

In order to fit the long lateral flow membrane (4) into a relatively small optics block (10-16), membrane (4) may optionally fold back (8) through a slot (7) in the plastic support (1), thus reducing the length of the test device.

The optics block (10-16) will normally consist of a support stage (10), an optional electrode (11) which can interface with optional sample and flow detection electrodes (9) present on the support (1), and typically an optics opening (12) through which the detection zone on the test device (6) may be observed by fluorescence or luminescence means. In the preferred fluorescence detection embodiment, light source (13), which may be a light emitting diode or the light, will have its excitation wavelength optimized by optional excitation filter (14). This excitation light passes through optics opening (12) and illuminates the detection zone (6) on the underside of the test strip. Fluorescence energy emitting from detection zone (6) is typically filtered by emission filter (15), and this in turn illuminates photodetector (16), which generates an electrical signal that is amplified as appropriate, digitized, and interpreted by the meter's microprocessor.

In use a sample, such as a drop of whole blood (20) is placed onto the application port. The sample passes through the port and hydrates the lateral flow membrane (21), as shown in FIG. 1B.

After the sample has been applied, the relative configuration of the buffer chamber and lateral flow membrane is altered so as to enable the reaction buffer to flow out of the buffer chamber (30) and into the lateral flow membrane (31), where, typically over a period of a few minutes, the buffer passes through the membrane, carrying the test analyte to the fluorescent detection conjugate (32), and subsequently pass the detection zone (33) where the analyte and fluorescent detection conjugate may bind. Unbound fluorescent detection conjugate and any remaining unbound sample are then carried past the detection zone and onto the end of the lateral flow membrane. (See FIG. 1C).

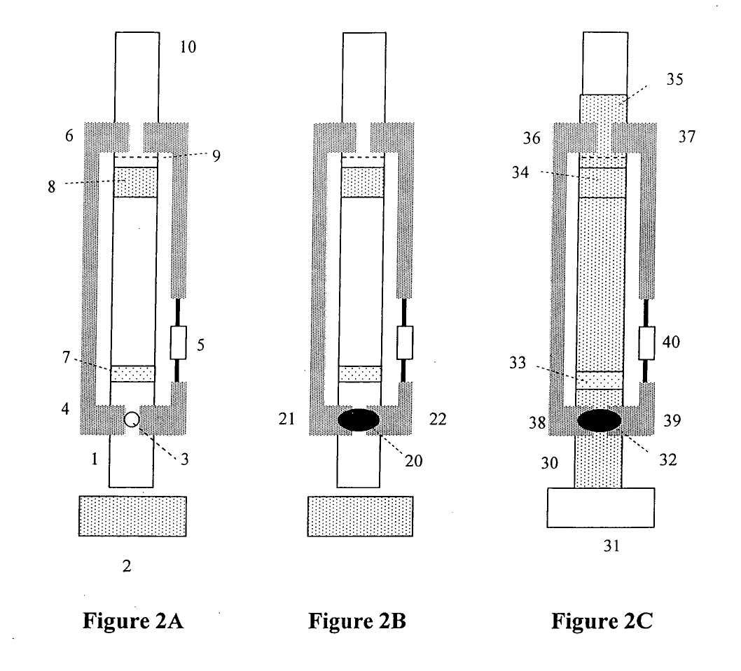

FIGS. 2A-2C describe how the electrical fluid detection elements of the present disclosure work in conjunction with sample application and transport buffer flow.

Here lateral flow membrane (1) and transport buffer chamber (2) are attached to a plastic support (not shown). This plastic support will contain sample application port (3). The device additionally contains conducting electrodes (4) arranged on either side of sample application port (3). These electrodes are electrically connected to a second set of conducting electrodes (6) by an electrical linkage through resistor (5). Typically these electrodes will then interface with electrodes on a meter's optics block (not shown). Lateral flow membrane (1) additionally contains a fluorescent detection conjugate (7) stored on the membrane in a dry form, and a detection zone (8) where analyte and bound fluorescent detection conjugate may bind. The lateral flow membrane (10) will typically extend beyond the detection zone (8) so that unbound fluorescent detection conjugate will be removed from the detection zone (8) by chromatography. The membrane may additionally contain an optional fold (9) to enable the membrane and optionally the electrodes to be folded back, thus reducing the total length of the assay device, and placing the detection zone closer to the end of the test device.

As shown in FIG. 2B, liquid sample (20) is normally placed on the membrane through a sample application port. This liquid sample will then reduce the resistance between the two electrodes on opposite sides of the sample application port (21), (22), causing an electrical resistance change, or other change in electrical properties, that may be detected by a meter that interfaces with the test device. This may be used to communicate beginning of reaction time, and sample size to the meter.

After sample has been applied, the relative configuration of the lateral flow membrane (30) and the transport buffer chamber (31) are altered to in a way that enables transport buffer to flow into the lateral flow membrane. The transport buffer flows past the sample port (32), and past the dry fluorescent detection conjugate (33) as shown in FIG. 2C. The transport buffer hydrates the fluorescent detection conjugate (33), and transports it and the analyte to the detection zone (34). There a fluorescent particle labeled detection antibody binds to an epitope on the analyte, and the analyte in turn binds to an immobilized capture antibody in the detection zone (34), typically by a second epitope. The transport buffer then continues wicking through the lateral flow membrane (35), passing “end of reaction” electrodes (36) and (37). When the transport buffer passes electrodes (36) and (37), a second drop in electrical resistance occurs in this electrode system. Since “end of reaction” electrodes (36) and (37) are partially isolated from “sample detection” electrodes (38), (39) by resistor (5), (40), the two signals may be distinguished.

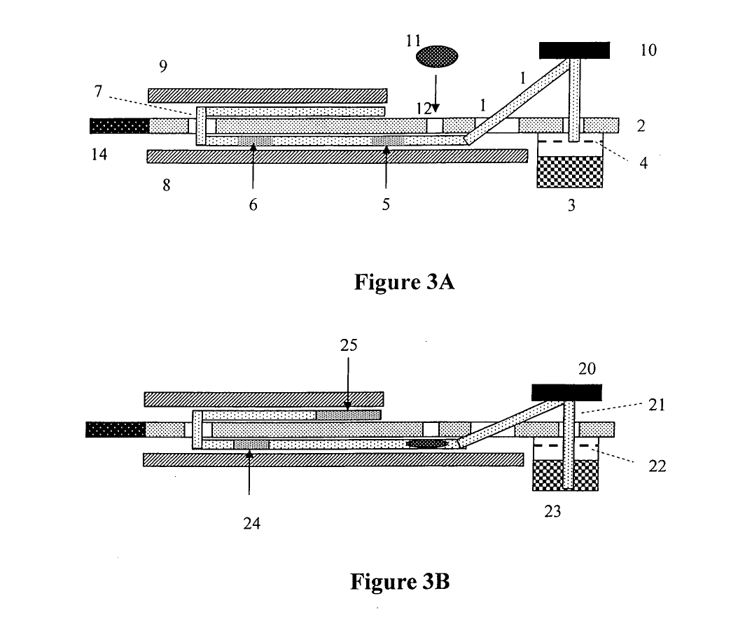

FIGS. 3A and 3B show an example of one mechanism to control the flow of transport buffer between the transport buffer chamber, and the lateral flow membrane. Here the lateral flow membrane (1) is again placed on plastic support (2). This support also contains a small chamber (3) containing a transport buffer. This chamber is sealed with a thin breakable covering (4). The lateral flow membrane is present in a continuous path, but may have selected portions covered with a plastic exoskeleton or plastic support in order to lend rigidity and strength when needed (not shown). The lateral flow membrane contains zones where fluorescent detection conjugate (5), and immobilized capture antibody (6) are present in a dry form. As before, the lateral flow membrane may fold back through a slot (7) in support structure (2) in order to reduce the length of the device. The lateral flow membrane will frequently be covered with one or more transparent plastic sheets (8), (9) to protect the lateral flow membrane from the outside environment. In this configuration, the device may also have a plastic button (10) attached to a plastic stiffener (not shown) that provides additional rigidity to this portion of the lateral flow membrane, and positions the membrane so that the application of force to button (10) will drive the lateral flow membrane through breakable covering (4) and into buffer chamber (3).

In FIG. 3A, sample (11) may be applied to the lateral flow membrane (1) through sample port (12). An additional slot in support plastic (13) enables the lateral flow membrane to pass from one side to of support structure (2) to the other side, and gives the membrane some flexibility of movement between the plunger up and plunger down states of the device. The device electrodes that enable the electrodes (shown in FIGS. 2A-2C) to interface with meter electrodes are present at the end of the device (14).

After sample (11) has been applied, the device is activated by pressing button (20). This forces the plastic sheathed end of the lateral flow membrane (21) past the breakable covering (22) and into the transport buffer chamber (23). The transport fluid then wicks down the length of the lateral flow membrane, carrying fluid past the fluorescent detection conjugate and depositing the fluorescent detection conjugate in the detection zone (24) if analyte is present. Unbound fluorescent detection conjugate (25) migrates to the end of the lateral flow membrane, as shown in FIG. 3B.

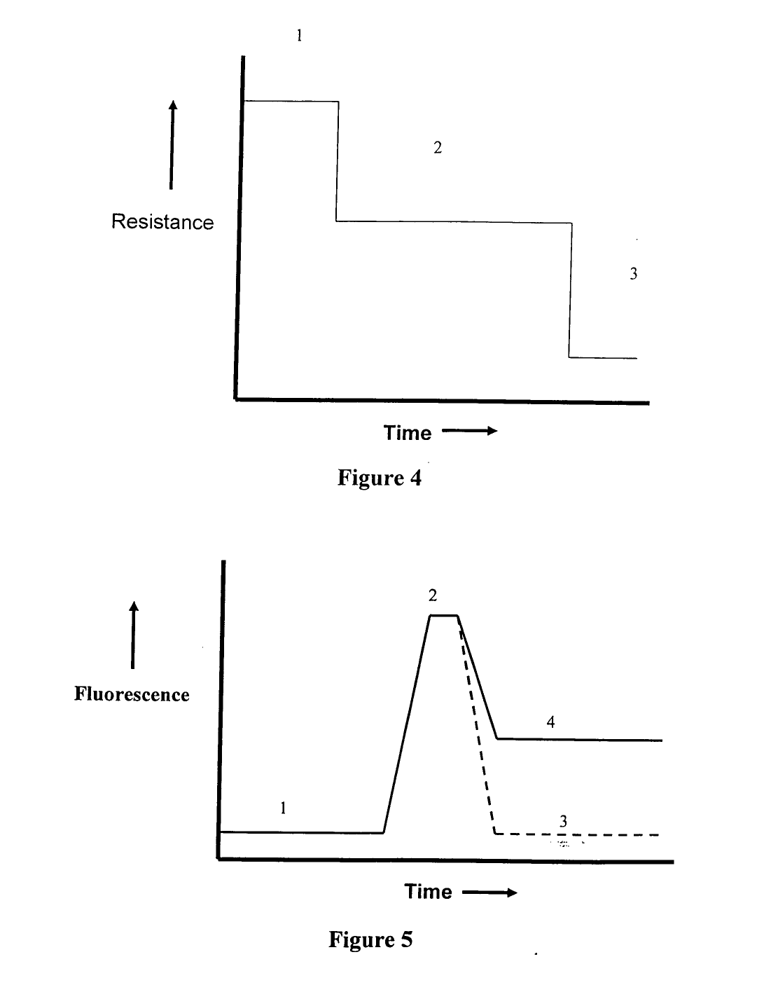

FIG. 4 shows an example of a graph of electrical resistance versus time for the electrode configuration previously discussed in FIG. 2. The resistance between the two electrodes is initially very high (1), but drops to an intermediate level upon application of sample to the fluid sensor electrodes (2). This intermediate level of resistance is kept at an intermediate level by a resistor (FIG. 2 (5)), which limits the resistance to an intermediate value even if the fluid sensor electrodes are exposed to a very low resistance sample. When the transfer buffer reaches the end of reaction electrodes, the resistance across this electrode pair is reduced, producing a still lower electrical resistance value (3).

This set of resistance “stair steps” can convey information to the meter that may be used by the meter's microprocessor to control reaction timing, and detect common user errors. The transition between the initial high resistance level (1) and the intermediate resistance level (2) lets the meter know the exact time that the sample was applied. Additionally, the magnitude of the resistance drop between (1) and (2) lets the meter know approximately how much sample was applied. If only a very small amount of sample was applied, then the resistance drop between (1) and (2) will be comparatively tiny. By contrast, if a large amount of sample is applied, the resistance drop between (1) and (2) will be limited only by the resistor (FIG. 2(5)).

Based upon this information, after the initial sample has been applied, the meter can then prompt the user to press the transport buffer button, and also warn the user if insufficient sample was applied. The meter also is triggered to start interrogating the detection zone using the meter's fluorescence detector.

The time lapse between the initial resistance drop (1), (2), and the final resistance drop when the transport buffer reaches the end of the reaction detection electrodes (2), (3) may be used to detect common user errors. If the user pressed the transport buffer button too late, then the time lag between the (1)-(2) drop and (2)-(3) drop will be unexpectedly short.

FIG. 5 shows a diagram of a typical fluorescence profile produced by the device. Initially after sample application, and while the transport buffer is migrating up the lateral flow membrane, the meter's fluorescence detector's observations of the detection zone will detect only a small, unchanging amount of background signal (1). As the front of fluorescent detection conjugate passes the detection zone, the fluorescence signal rises rapidly, eventually reaching a peak as the front passes directly by the fluorescence detector (2). If no analyte is present, no fluorescent detection conjugate will bind to the capture antibody immobilized in the detection zone, and the fluorescent signal will return to a value close to the original baseline value (3). By contrast, if analyte is present, it will bind to the immobilized capture antibody in the detection zone, and in turn the fluorescent detection antibody will bind to the analyte, producing a persistent fluorescent signal (4) that continues at a level above that of the baseline even after the main front of unbound fluorescent conjugate has passed. The difference in intensity between the persistent fluorescent signal (4) and the expected no-analyte baseline (3) is proportional to the amount of analyte present.

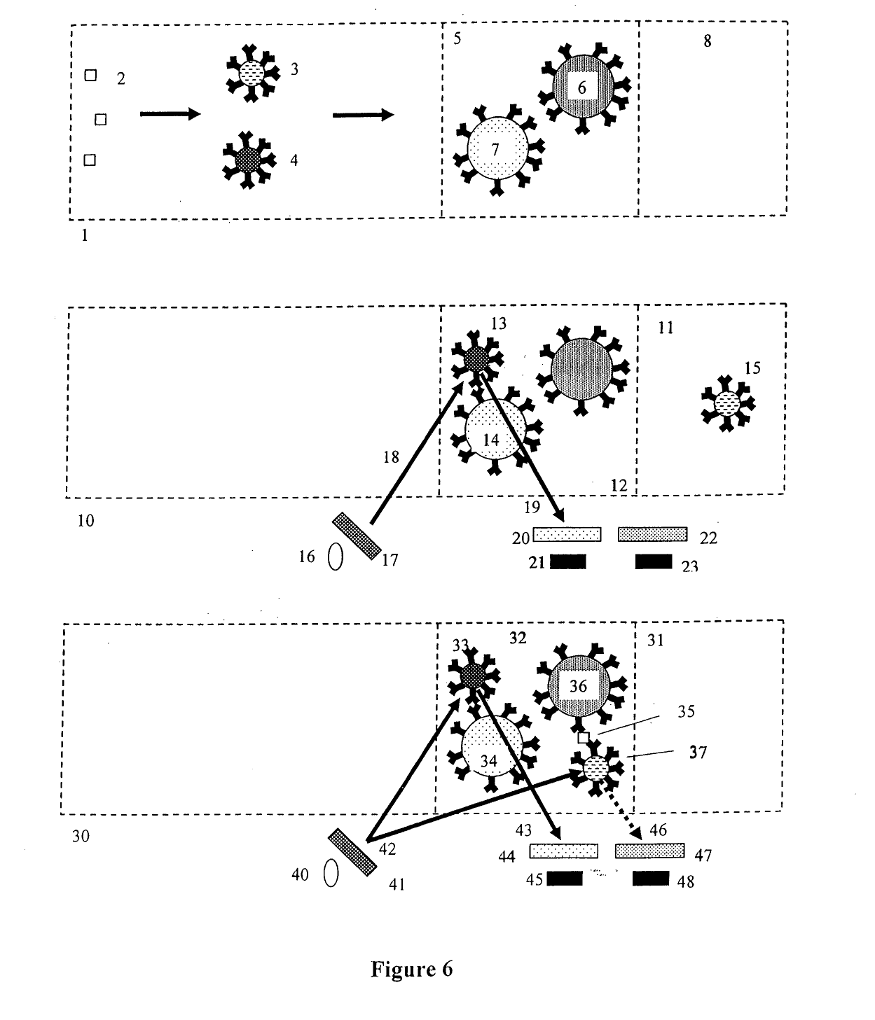

FIG. 6 shows how an internal control may be built into the device that may be read by a dual wavelength fluorescence detector. In this figure, a lateral flow membrane (1) is exposed to a test analyte (2). This test analyte is carried by the reaction buffer (not shown) to fluorescent particles (3) coupled with an antibody against a first epitope on the test analyte. These particles will normally be excited by a first wavelength, and emit fluorescence energy at a second wavelength. Also present are a set of control fluorescent particles (4) coupled with a control antibody that specifically reacts with a control protein. This control fluorescent particle may be exited by the same first wavelength used to excite the anti-test analyte fluorescent particles, but should optimally emit fluorescent energy at a third wavelength that is distinct from the first and second wavelengths. Lateral flow device (1) will additionally contain a detection zone (5) containing both immobilized antibodies (6) against a second epitope on the test analyte, and an immobilized control protein (7) that binds to the control fluorescent particles (4). In this example, antibodies (6) and control protein (7) are immobilized by binding the antibodies and control protein to larger latex beads that have a size too large to migrate through the meshwork of the lateral flow membrane. In alternative schemes, however, antibodies (6) and control protein (7) may be directly bound to the detection zone of the lateral flow membrane.

Lateral flow membrane (1) will additionally contain an “end” region (8) beyond the detection zone (5) where unbound particles (3) and (4) will normally migrate due to capillary action of the transport buffer if binding to the antibodies (6) or control protein (7) does not occur.

The case where there is no test analyte present, but the control particles indicate that no deterioration of test components has occurred, is shown in (10). Here the transport buffer has previously been released, and all mobile test elements have either been transported to the “end” region of the lateral flow membrane (11), or have become bound to the detection zone (12). In this situation, since no deterioration of test components has occurred, the control particles (13) containing a control antibody reactive to a control protein (14), bind to the control protein (14), and thus remain immobilized in the detection zone (12). By contrast, the fluorescent particles containing an antibody reactive to the test analyte (15), have nothing to bind to, and thus migrate to the “end” (11) of the lateral flow membrane, where they pass out of the detection zone, and thus are not observed by a fluorescence-measuring detector.

In this example, the detection zone is monitored by a fluorescence detector containing a light source (16) and emission filter (17). This transmits excitation light energy at a first wavelength (18) to the detection zone (12). Fluorescent energy emitted by the control particles (13) (19) passes through a wavelength filter selected to transmit the wavelengths of this particle (20) and is detected by control photodetector (21).

By contrast, since there is no test analyte present, no fluorescent particles reactive to the test analyte (15) are present in the detection zone. As a result, no fluorescent light energy specific to test analyte detecting particles (15) is available to produce energy capable of passing through filter (22) selected to transmit the wavelengths of test analyte detecting particle (15), and thus no energy is detected by test analyte detecting photodetector (23).

The case where test analyte is present is shown by lateral flow membrane (30). Here again, the transport buffer has previously been released, and all mobile test elements have either been transported to the “end” region of the lateral flow membrane (31), or have become bound to the detection zone (32). In this situation, since again, no deterioration of test components has occurred, the control particles (33) containing a control antibody reactive to a control protein (34), bind to the control protein (34), and thus remain immobilized in the detection zone (32).

However since test analyte (35) is also present, analyte (35) will bind to the immobilized anti-analyte antibody present in the detection zone (36). Additionally, fluorescent detection particle (37) that contains antibodies reactive with a different epitope on analyte (35) can also bind, forming a sandwich structure, that acts to immobilize detection particle (37) in detection zone (32).

As a result of this immobilization, both the control fluorescent particle and the analyte specific fluorescent particle can now be detected. Assuming that the two particles emit at different wavelengths, the relative levels of the two particles can then be determined.

To do this, the detection zone is observed by a dual wavelength fluorescence detector as before. Here light source (40) sends excitation energy through excitation filter (41) resulting in excitation energy of a first wavelength (42) to illuminate the detection zone (32). This energy excites both the control fluorescent particles (33) and the analyte detecting fluorescent particles (37). The control particles emit fluorescent energy at the control wavelength (43), which passes through control filter (44) and is detected by control photodetector (45). The analyte detecting particles emit fluorescent energy at an alternative wavelength (46), which passes through analyte wavelength filter (47) and is detected by analyte photodetector (48).

The information from the control photodetector and the analyte photodetector is then passed to the instrument's microprocessor. There, the analyte signal may be translated into a measurement that determines the relative level of analyte present, and the control signal may be translated into a fitness for use determination. For example, if there is no analyte present, and also no control signal present, the microprocessor can determine that probable reagent deterioration or analytical error occurred, and transmit an error measurement to the user, rather than an inappropriate “low analyte” value. Conversely, if the analyte value was low, but the control signal was adequate, the instrument can determine that the low analyte value is probably accurate, and transmit the computed “low analyte” value to the user without any warning or error message.

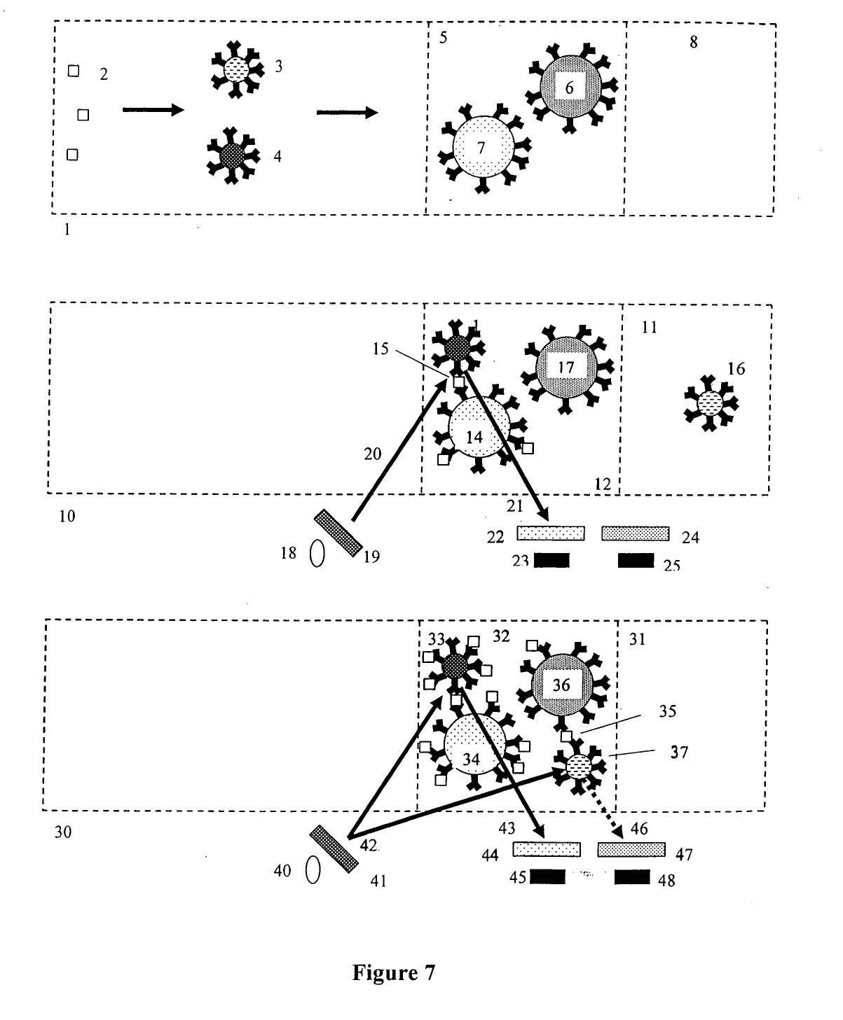

FIG. 7 shows how fluorescent particles coupled with low and high affinity antibody against a target analyte may be used to extend the dynamic range of an assay. Typically, sandwich based immunoassays will suffer from a “hook” effect in which, at high levels of analyte, the analyte will saturate the binding sites on both the capture antibody portion of the sandwich, and the detector antibody portion of the sandwich, leading to an inhibition of binding between the capture antibody and detector antibody.

Again, in this figure, a lateral flow membrane (1) is exposed to a test analyte (2) that may span a broad dynamic range between trace levels of the analyte, and saturating levels of the analyte. This test analyte is carried by the reaction buffer (not shown) to high sensitivity fluorescent particles (3) coupled with a high affinity antibody against a first epitope on the test analyte. These particles will normally be excited by a first wavelength, and emit fluorescence energy at a second wavelength. Also present are a set of low sensitivity fluorescent particles (4) coupled with a low affinity antibody that specifically reacts with the test analyte. This low affinity fluorescent particle may be exited by the same first wavelength used to excite the high sensitivity anti-test analyte fluorescent particles, and may either emit at the same wavelength as the high sensitivity particle, or optionally emit fluorescent energy at a third wavelength that is distinct from the high affinity particle to better distinguish the low analyte concentration part of the response curve from the high sensitivity part of the response curve. As before, lateral flow device (1) will additionally contain a detection zone (5) containing both high affinity immobilized antibodies (6) against a second epitope on the test analyte, and low affinity immobilized antibodies against a second epitope on the test analyte (7). In this example, both high affinity antibodies (6) and low affinity antibodies (7) are immobilized by binding the antibodies and control protein to larger latex beads that have a size too large to migrate through the meshwork of the lateral flow membrane. In alternative schemes, however, high affinity antibodies (6) and low affinity antibodies protein (7) may be directly bound to the detection zone of the lateral flow membrane.

Lateral flow membrane (1) will additionally contain a “end” region (8) beyond the detection zone (5) where unbound particles (3) and (4) will normally migrate due to capillary action of the transport buffer if binding to the high or low affinity antibodies (6), (7) does not occur.

The case where only small amounts of analyte are present is shown in (10). Here the transport buffer has previously been released, and all mobile test elements have either been transported to the “end” region of the lateral flow membrane (11), or have become bound to the detection zone (12). In this situation, since there is only a small amount of analyte present, the fluorescent particles containing the high affinity antibodies (13) bind to the small amounts of target analyte (15) bound to the immobilized antibody (14), and thus remain immobilized in the detection zone (12). By contrast, the fluorescent particles containing a low affinity antibody reactive to the test analyte (16), will tend to lose any low amounts of test analyte to the high affinity antibody, fail to bind to the low affinity immobilized antibodies (17), and thus migrate to the “end” (11) of the lateral flow membrane, where they pass out of the detection zone, and thus are not observed by a fluorescence measuring detector.

In this example, the detection zone is monitored by a fluorescence detector containing a light source (18) and emission filter (19). This transmits excitation light energy at a first wavelength (20) to the detection zone (12). Fluorescent energy (21) emitted by the high affinity fluorescent detection particles (13) passes through a wavelength filter selected to transmit the wavelengths of this particle (22) and is detected by either a single photodetector, or alternatively a photodetector assigned to detect low concentrations of the analyte (23).

By contrast, since the low affinity antibodies have lost any low amounts of target analyte present to the high affinity antibodies, very few low-affinity fluorescent particles reactive to the test analyte (16) are present in the detection zone. As a result, little fluorescent light energy specific to the low affinity test analyte detecting particles (16) is available to produce energy capable of passing through filter (24) selected to transmit the wavelengths of the low affinity (high concentration detecting) test analyte detecting particle (16), and thus little energy is detected by high concentration test analyte detecting photodetector (25). Note that in an alternative configuration, the two types of particles may emit the same wavelength, and be detected by a single filter/photodetector combination.

The case where high levels of test analyte are present, which in this example are nearly saturating the high affinity antibodies, is shown by lateral flow membrane (30). Here again, the transport buffer has previously been released, and all mobile test elements have either been transported to the “end” region of the lateral flow membrane (31), or have become bound to the detection zone (32). In this situation, the high affinity fluorescent particles (33) are almost fully saturated with test analyte, and thus will start to become unstuck from the immobilized antibody if the levels of test analyte increase still further, are shown still binding to immobilized high affinity antibody (34). In this example, these remain in the detection zone (32), but will become unbound if analyte levels increase much further.

Since high levels test analyte (35) are present at concentrations that are about to saturate the high affinity antibodies, analyte (35) will now bind to the low affinity immobilized anti-analyte antibody present in the detection zone (36). Additionally, fluorescent detection particle (37) that contains low affinity antibodies reactive with a different epitope on analyte (35) can also bind, forming a sandwich structure, that acts to immobilize low affinity (high concentration detecting) detection particle (37) in detection zone (32).

As a result of this immobilization, both the high affinity fluorescent particle and the low affinity analyte specific fluorescent particle can now be detected. Assuming that the two particles emit at different wavelengths, the difference between very low levels of analyte, and very high (saturating) levels of analyte can then be determined.

To do this, the detection zone is observed by a dual wavelength fluorescence detector as before. Here light source (40) sends excitation energy through excitation filter (41) resulting in excitation energy of a first wavelength (42) to illuminate the detection zone (32). This energy excites both the high affinity fluorescent particles (33) and the low affinity analyte detecting fluorescent particles (37). The high particles emit fluorescent energy at the control wavelength (43), which passes through control filter (44) and is detected by the low analyte concentration detecting photodetector (45). The low affinity analyte detecting particles emit fluorescent energy at an alternative wavelength (46), which passes through analyte wavelength filter (47) and is detected by the high analyte concentration detecting photodetector (48).

The information from the low concentration detecting photodetector and the high concentration detecting photodetector is then passed to the instrument's microprocessor. There, the analyte signals may be translated into a measurement that determines the relative level of analyte present over a broad dynamic range. If, for example, the high-end detector is registering a signal, and the low end of the detector is not, the probable cause is the presence of analyte that has saturated the high sensitivity antibodies. This can prevent the instrument from mistaking extremely high levels of analyte for extremely low levels, and help provide good accuracy over an extended dynamic range.

### EXAMPLE 1

### Construction of Test Strips

Prototype test strips were constructed using Fusion 5 membrane (Whatman Corporation), 10-mil thick transparent polycarbonate, 10 ml thick white polystyrene, 415 double-sided adhesive (3M corporation), and 1 mil aluminum foil with an adhesive backing.

To construct the device, a 5/16″ wide×5 1/16″ long piece of Fusion 5 membrane was cut. The sample application zone was located ¾″ from one end. A conjugate storage zone was located 1⅜″ away from the same end. The detection zone was a ¼″ wide zone located 2¼″ away from the same end. A fold, used to bend the Fusion 5 in a 180° turn was located 3/16 away from this end. The remainder of the Fusion 5 membrane was used as a wick to remove excess buffer and unbound fluorescent detection particles from the observation zone.

To hold the test strip, white polystyrene was covered with a layer of 415 adhesive. The styrene strip was ¾″ wide, and 3″ long. A ⅜″ wide slot× 1/32″ deep was cut in the center of the polystyrene exactly 5/16″ away from the end of the strip, parallel to the narrow (¾″) side of the polystyrene. Two thin (⅛″ wide) strips of aluminum foil were adhered to polystyrene with adhesive, forming two conductive paths that went from one end of the polystyrene to the other end along the long (3″) axis. These strips of aluminum foil contained three electrode structures that protruded out from the central ⅛″ wide conductive strip. One protruding electrode structure extended further into the body of the polystyrene strip, forming two electrodes exactly underneath the sample application hole (which was cut ⅜″ away from the opposite end of the polystyrene from where the slot was cut). These are used to detect the first resistance drop caused by sample application. Another set of protruding electrodes was designed to wrap around the 10 mil thick layer and adhere to the other side of the polystyrene strip. These electrodes are used to detect the resistance drop caused by the transport buffer finally migrating to the end of the test strip. At the far end of the test strip (near the ⅜″ wide slot), the two aluminum foil strips were widened to ⅜″ wide, and allowed to wrap around the ¾″ wide end of the test strip, forming a contact surface that enables the polystyrene test strip to make electrical contact with meter electrodes.

The Fusion 5 membrane was threaded through this slot, and bent at the fold, creating a “U” shaped membrane that bent back on itself. This was then stuck to the center of the test strip using the 415 adhesive.

To protect the Fusion 5 membrane from damage, and to allow the transport buffer to flow up the membrane in an undisturbed manner, two guard ribs with length 2 13/16″×⅛″ wide made from 10 mil polystyrene with 415 adhesive on either side were placed on opposite sides of the Fusion 5 membrane, separated by an air gap from actual contact with the membrane. On top of the membrane and guard ribs is then placed a 2 13/16″ long×¾″ wide of transparent 10-mil polycarbonate, with a layer of transparent 415 adhesive on the side facing the Fusion 5 membrane. The net effect is to create a sandwich structure with a long narrow strip of Fusion 5 membrane placed in between a reflective layer of white polystyrene on one side, and which may be viewed through the transparent polycarbonate and 415 layer on the other side. This may be seen in more detail in FIGS. 1A-1C.

In order to electrically distinguish signals from the first, sample detecting electrode from the second, end of reaction electrode, a small break is made in the ⅛″ wide connecting foil running between the electrodes on one side, and a 150,000 ohm resistor placed in between. This configuration is shown in more detail in FIGS. 2A-2C.

In use, the test strip was held down into a combination fluorescence detector and electrical signal detector (previously described in Zweig S E, Meyer B G, Sharma S, Min C, Krakower J M, Shohet S B: Membrane-based, dry-reagent prothrombin time tests. Biomed Instrum Technol. 30(3): 245-56(1996); and U.S. Pat. No. 5,554,531 and D371,605. The test strip was held in place by a test strip holder (previously described in US patent D438,971).

### EXAMPLE 2

### Dye Migration

In this experiment, 5 microliters of a 1 mg/ml solution of Blue dye #1 in water was applied to the conjugate release zone of the test strip, which is located 1⅜″ away from the end of the strip that protrudes slightly out of the plastic sandwich. The blue dye was applied as a thin strip and allowed to dry. After drying, a solution of buffered 0.9% sodium chloride in water was applied to the end of the strip, and dye migration observed. It was seen that the blue dye migrated up to the fold of the strip, and then went through the slot (disappearing from view through the plastic side), and then migrated to the end of the test strip, migrating as a coherent well-defined colored band during this time. The time elapsed from start to completion was about 4 minutes, and about 1 ml of buffer was consumed during this process.

### EXAMPLE 3

### Electrical Resistance Tests

Test strips were made as discussed previously, and placed into the combined fluorescence detection and electrical resistance detection optics block described previously. This optics block measured electrical resistance from about 20,000 ohms to about 800,000 ohms, and presented the results as a digitized 12 bit (0-4095) signal in this region. Studies with application of 10-ul whole blood, as well as application of a buffered 0.9% sodium chloride transport buffer, produced a stair step resistance pattern as exemplified in FIG. 4.

### EXAMPLE 4

### Immunochemical Binding Experiments

Capture microbeads: 1.04 micron diameter carboxyl modified latex microbeads were obtained from Bangs Labs. Monoclonal human chorionic gonadotropin antibody E20106 was obtained from Biodesign Corporation. 400 micrograms of antibody were covalently coupled to 12.5 mg of beads using a commercially available conjugation kit (Polysciences Corporation).

After conjugation, the beads were blocked using the Polysciences blocking buffer, and resuspended to a concentration of approximately 1.5% solids for use.

Microbeads have a high tendency to aggregate, which reduces the effective surface area of the beads, and diminishes the sensitivity of the assay. To improve sensitivity, the capture beads were placed in a 5% Trehalose, 1 mg/ml BSA, 0.1% Tween-20, 1 mg/ml Blue-dye #1 solution in 20 mM phosphate buffer pH 7.2, and were sonicated before use. These sonicated beads were then applied to the Fusion 5 membrane using a low volume air brush (approximately 10 ul of a bead solution per inch of membrane), followed by air-drying. The beads were applied to both sides of the membrane, in a roughly ¼″ wide stripe, which was approximately the same width as the diameter of the spot observed by the instrumented fluorescence reader described previously.

Fluorescent detection particles. Monoclonal human chorionic gonadotropin antibody 77F12 was obtained from HiTest Corporation, and covalently conjugated to 655 nm quantum dots using a commercially available quantum dots antibody conjugation kit (Quantum Dots corporation, Hayward Calif.). Quantum dots are small (roughly 10 nanometer diameter) spherical particles with a central core (roughly 5 nanometers in diameter) containing a highly fluorescent metallic complex that emits high intensity fluorescent light, tuned to a relatively narrow emission band (in this case 655 nanometers) upon receiving shorter wavelength excitation energy.

Here, antibody conjugated 655 nm quantum dots, at a concentration of approximately 1.5 micromolar, were treated with Blockaid™ blocking solution (Molecular Probes corporation) and briefly sonicated to reduce aggregation, and 5 ul of solution was spotted onto the Fusion 5 membrane approximately 1.25 inches away from the capture bead line.

In use, 10 ul of whole blood was spiked with hCG to the final equivalent values of 0, 2.5, 5, 10, 20, and 40 international units/ml whole blood of human chorionic gonadotropin (Sigma-Aldrich Corporation), and these blood samples were applied to a series of Fusion 5 membranes with the fluorescent anti-hCG 655 nm quantum dots fluorescent detection particles, and the monoclonal antibody coupled microspheres as discussed above. Immediately after spotting, the Fusion5 membrane was exposed to a running buffer consisting of 150 mM NaCl, 10 mM Phosphate Buffer, ph 7.2, which additionally had 1 mg/ml BSA to prevent nonspecific sticking of the hCG sample to the Fusion5 membrane. The red cells present in the whole blood sample remained trapped in the meshwork of the membrane, while the transport buffer transported the hCG analyte past the fluorescent detection particles, mobilized the detection particles, and transported the mobile fluorescent detection particle (with bound hCG) past the stationary capture antibody bound microbeads. The microbead size (1 micron) is large enough that the beads are captured by the meshwork of the membrane, and are thus rendered immobile.

In this experiment, the flow of the fluorescent particles and the extent of binding to the stationary capture beads in the detection zone was monitored by illuminating the entire membrane with 500 nm excitation light, produced by putting a 500 nm+/−10 nm bandpass filter (Edmund Optics) in front of a standard slide projector. The fluorescent signal was detected by digital photography, using a 550 nm cutoff longpass filter (Edmund Optics) in front of a Nikon Coolpix 995 digital camera. The resulting digital image was downloaded into a standard computer, and the regions on the membrane corresponding to the detection zone were examined.

Under these conditions, it was found that the approximate sensitivity of the whole-blood hCG assay was approximately 5 international units (IU) of hCG per ml of whole blood.

### EXAMPLE 5

### Enhancing the Migration of Particles within the Matrix of a Porous Bibulous Membrane

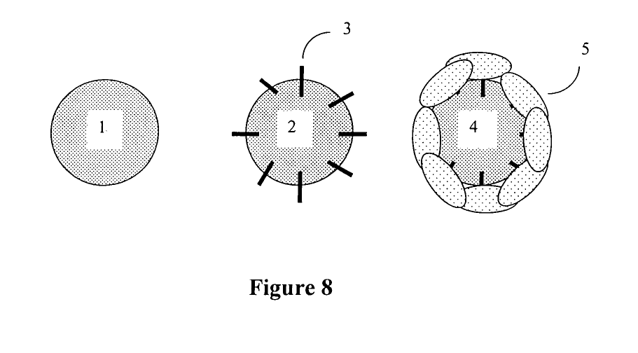

Although certain particles, such as quantum dots, may have an intrinsically favorable surface chemistry so that, if the diameter of the particle is sufficiently smaller than the average pore size of the matrix of the bibulous membrane, the migration of the particle, as carried by a suitable aqueous solvent, proceeds without unfavorable distortion brought about by particle-particle aggregation, or sticking to the matrix, in other cases, the surface chemistry of the particle may be intrinsically less favorable. In these latter cases, the surface chemistry of the particle may be modified, either through deliberate chemical modification, or by selecting for particles with intrinsically favorable surface chemistry, so as to minimize particle aggregation or sticking to the matrix. As an example, particles with problematic hydrophobic regions, rendering these particles prone to aggregation by hydrophobic interactions, may have these hydrophobic regions removed by chemical modification. Similarly, particles with charged regions, rendering them susceptible to interaction with oppositely charged regions on other particles, or on the matrix, may also have these charged groups removed by chemical modification. One simple way to do this is shown in FIG. 8. FIG. 8(1) shows a particle that has desirable fluorescent or luminescent properties, and would otherwise be suitable for the present invention, but which otherwise shows unfavorable aggregation or membrane matrix interactions. To overcome this problem, the particle is linked to a ligand by a covalent linkage, or by tight non-covalent linkage such as an avidin-biotin linkage. This is shown in FIG. 8(2), and the ligand is shown as FIG. 8(3). After this is done, a second type of molecule, such as biotinated bovine serum albumin, is added. This second molecule (5) binds to the ligands on the particle (seen as FIG. 8(4)), and can prevent the hydrophobic or charged groups on the particle from interacting with other particles, or with the membrane matrix.

### EXAMPLE 6

Resistance and Fluorescent Data

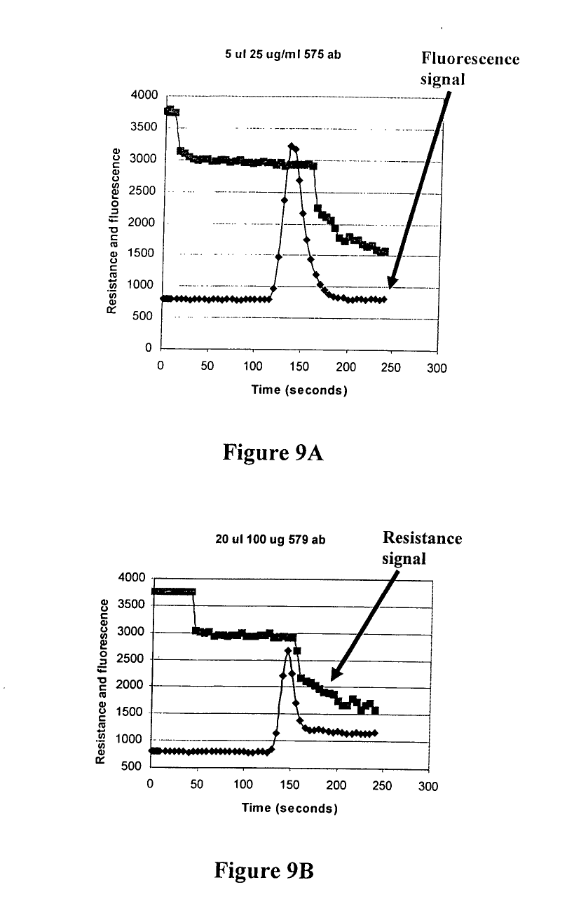

Initial immunochemical binding experiments. In this experiment, the strip configuration discussed in previous examples (e.g., examples 2 and 3) was evaluated with a simple binding experiment in which strepavidin conjugated 555 nm quantum dots were allowed to migrate towards stationary 1 micron diameter sized strepavidin conjugated microbeads in the presence or absence of various amounts of biotinated antibody. In this case, when biotinated antibody was present at higher levels, the quantum dots were bound to the stationary 1-micron diameter beads, and when the biotinated antibody was present at lower levels, no such binding occurred. The test strips were monitored in a hand held fluorimeter that performed simultaneous resistance and fluorescence measurements. The results clearly showed that at low levels of antibody, the fluorescent quantum dots migrated past the stationary 1-micron diameter microbeads without binding. However at higher levels of antibody, the quantum dots became immobilized to the microbeads, and a persistent fluorescent signal resulted. Note that this fluorescence change occurred in the time period between the first resistance drop due to application of transport buffer, and the second resistance drop indicating that the transport buffer was beginning to reach the end of the test strip. Resistance and fluorescent data are shown in FIGS. 9A and 9B.

### EXAMPLE 7

### Dose Response Curve in Response to Varying Levels of hCG

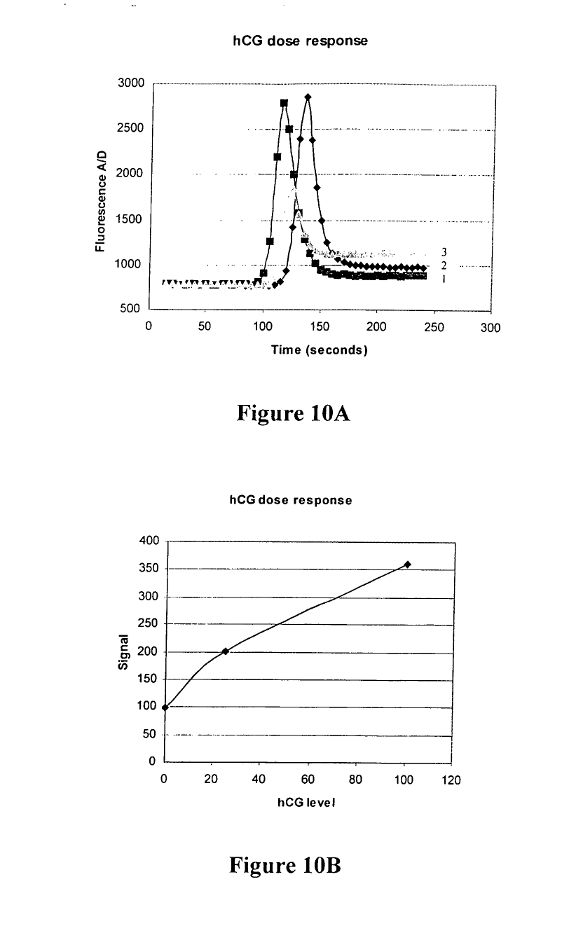

This example shows an experiment in which the previously discussed test strip design may be used to quantitatively distinguish between different levels of hCG in a liquid sample. In this experiment, a first monoclonal anti-hCG antibody was covalently coupled to 1-micron latex beads. A second monoclonal anti-hCG antibody was biotin conjugated. Strepavidin conjugated 555 nm quantum dots were placed midway between the two. The samples were air dried, and processed into test strips as previously described. The test strips were then challenged with various levels (0, 25, 100 ug/ml) of hCG dissolved in Phosphate Buffered Saline, ph 7.0, with 1 mg/ml BSA. The strips were then run on a portable hand-held fluorimeter, and the fluorescence profiles obtained. The results are shown in FIG. 10(A). As may be seen, at low levels of hCG (1), the retained fluorescence is low. At higher levels of hCG (2), the retained fluorescence is higher, and at the highest levels of hCG (3), a still higher level of retained fluorescence is obtained. FIG. 10(B) shows that the level of retained fluorescence may be used to construct a dose response curve.

It is understood that the foregoing detailed description and accompanying examples are merely illustrative, and are not to be taken as limitations upon the scope of the invention. Various changes and modifications to the disclosed embodiments will be apparent to those skilled in the art. For example, many of the assay formats disclosed in the following patents and patent application publications may be used or be modified to be used in the present devices and methods U.S. Pat. No. 5,602,040; 5,622,871; 5,656,503; 6,187,598; 6,228,660; 6,818,455; No. 2001/0008774; 2005/0244986; U.S. Pat. No. 6,352,862; No. 2003/0207465; 2003/0143755; 2003/0219908; U.S. Pat. Nos. 5,714,389; 5,989,921; 6,485,982; Ser. No. 11/035,047; U.S. Pat. Nos. 5,656,448; 5,559,041; 5,252,496; 5,728,587; 6,027,943; 6,506,612; 6,541,277; 6,737,277 B1; U.S. Pat. Nos. 5,073,484; 5,654,162; 6,020,147; 4,956,302; 5,120,643; 6,534,320; 4,942,522; 4,703,017; 4,743,560; 5,591,645; and RE 38,430 E. U.S. patents and publications referenced herein are incorporated by reference.

1. A device for detecting an analyte in a sample, which device comprises:
a naturally hydrophilic membrane comprising a first location and a second location, wherein
said first location comprises a dried first fluorescent or luminescent particle labeled binding reagent capable of binding to an analyte, if present in a sample to be tested, to form a first complex comprising said first labeled binding reagent and said analyte;
said second location, downstream from said first location, comprises an immobilized second binding reagent capable of binding to said first complex, if present, to form a second complex comprising said first labeled binding reagent, said analyte and said immobilized second binding reagent;
a liquid for transporting said analyte and said first labeled binding reagent to said second location to form said second complex,
whereby the presence, absence and/or amount of said analyte in said sample is determined by assessing fluorescence or luminescence comprised in said second complex at said second location.

2. The device of claim 1, wherein the naturally hydrophilic membrane comprises polymer treated glass fiber.

3. The device of claim 1, wherein the naturally hydrophilic membrane is SLF5 membrane.

4. The device of claim 1, wherein the device comprises a single naturally hydrophilic membrane.

5. The device of claim 1, wherein said first location, said second location or both locations are in the form of a zone or zones.

6. The device of claim 1, wherein the fluorescent particle comprises a quantum dot.

7. The device of claim 1, wherein the fluorescent or luminescent particle labeled binding reagent is air dried or lyophilized.

8. The device of claim 1, wherein the fluorescent or luminescent particle labeled binding reagent is dried in the presence of a material that:
a) stabilizes the fluorescent or luminescent particle labeled binding reagent;
b) facilitates resuspension of the fluorescent or luminescent particle labeled binding reagent in the liquid; and/or
c) facilitates mobility of the fluorescent or luminescent particle labeled binding reagent.

9. The device of claim 8, wherein the material is a protein, a peptide, a polysaccharide, a sugar, a polymer, a gelatin or a detergent.

10. The device of claim 1, wherein said first labeled binding reagent, said second immobilized binding reagent or both binds specifically to the analyte.

11. The device of claim 1, wherein neither the first labeled binding reagent nor the second immobilized binding reagent binds specifically to the analyte, and a scavenger substance is used to improve detection specificity.

12. The device of claim 1, wherein said first labeled binding reagent, said second immobilized binding reagent or both is an antibody to the analyte.

13. The device of claim 1, wherein the second binding reagent is immobilized at the second location by absorption, adsorption, or covalent binding to the naturally hydrophilic membrane; or attached to another substance or particle that is immobilized to the naturally hydrophilic membrane.

14. The device of claim 1, wherein a sample liquid alone is used to transport the analyte and the labeled binding reagent to the second location.

15. The device of claim 1, wherein a developing liquid is used to transport the analyte and the labeled binding reagent to the second location.

16. The device of claim 1, wherein said naturally hydrophilic membrane is supported by a solid backing.

17. The device of claim 16, wherein the membrane extends to the opposite side of the backing.

18. The device of claim 16, which further comprises a housing that covers at least the first and second locations on the membrane, wherein the housing comprises a sample application site to allow sample application upstream from or to the first location on the membrane and an opening around the second location to allow fluorescence or luminescence detection at the second location.

19. The device of claim 18, wherein the housing comprises a plastic material.

20. The device of claim 18, which further comprises a liquid holder and a means for transporting a liquid in the liquid holder to the first location or upstream from the first location on the membrane.

21. The device of claim 20, wherein the liquid is transported from the liquid holder to the membrane by a relative position change between the liquid holder and the membrane.

22. The device of claim 18, which further comprises a pair of electrodes on the membrane, wherein the pair of electrodes are spatially separated at the sample application site and at a site downstream from the second location on the membrane such that sample application and the liquid flow to the second location may be electrically monitored.

23. The device of claim 16, which further comprises a sample receiving member upstream from and in fluid communication with the first location, wherein sample receiving member is supported by the solid backing, and the sample is applied to the sample receiving member and then transported to the first and second locations sequentially.

24. The device of claim 23, which further comprises a housing that covers the first and second locations on the membrane and at least a portion of the sample receiving member, wherein the housing comprises an opening around the second location to allow fluorescence or luminescence detection at the second location.

25. The device of claim 24, wherein at least a portion of the sample receiving member is not covered by the housing and a sample is applied to the sample receiving member portion outside the housing and then transported to the first and second locations sequentially.

26. The device of claim 24, wherein first and second locations on the membrane and the sample receiving member are entirely covered by the housing, and the housing comprises a sample application site to allow sample application to the sample receiving member.

27. The device of claim 1, which further comprises, at the second location on the membrane, an additional immobilized binding reagent capable of binding to a second fluorescent or luminescent label, wherein the binding between the additional binding reagent and the second fluorescent or luminescent label is independent of the binding between the analyte in the sample and its respective immobilized binding reagent at the second location, and the two fluorescent or luminescent signals at the second location may be detected at two distinct spectra without interference from each other.

28. The device of claim 1, which comprises two different fluorescent or luminescent particle labeled binding reagents capable of binding to a first epitope of an analyte at the first location, wherein one labeled binding reagent has low binding affinity to the analyte and the other labeled binding reagent has high binding affinity to the analyte;
two different immobilized binding reagents capable of binding to a second epitope of the analyte, wherein one immobilized binding reagent has low binding affinity to the analyte and the other immobilized binding reagent has high binding affinity to the analyte;
at low analyte concentration, a sandwich of the analyte with the two low affinity antibodies is formed and at high analyte concentration, a sandwich of the analyte with the two high affinity antibodies is formed; and
two different fluorescent or luminescent particle labels may be detected at the same or different spectra.

29. The device of claim 1, which further comprises a control location downstream from, but in fluid communication with, the second location, wherein the control location comprises means for indicating a valid test result.

30. The device of claim 1, which further comprises a liquid absorption pad downstream from, but in fluid communication with, the second location.

31. A method for detecting an analyte in a sample, comprising
a) contacting a sample with the device of claim 1, wherein the sample is applied to a site of the membrane upstream of the second site;
b) transporting an analyte, if present in said sample, and the dried first fluorescent or luminescent particle labeled binding reagent capable of binding to an analyte, by a liquid to the second location to form the second complex comprising said first labeled binding reagent, said analyte and said immobilized second binding reagent at said second location; and
c) determining the presence, absence and/or amount of said analyte in said sample by assessing fluorescence or luminescence comprised in said second complex at said second location.

32. The method of claim 31, wherein the sample is applied to the first location of the membrane, or the sample is applied to a site of the membrane upstream of the first location of the membrane.

33. The method of claim 31, wherein the sample is whole blood, a serum, a plasma or a urine sample.

34. The method of claim 31, wherein the analyte is selected from the group consisting of a cell, a virus and a molecule.

35. The method of claim 31, wherein the analyte is selected from the group consisting of hCG, hLH, hFSH, hTSH, an antigen of an infectious organism, an antibody to an infectious organism and a disease marker.

36. The method of claim 31, wherein a sample liquid alone is used to transport the analyte and the labeled binding reagent to the second location.

37. The method of claim 31, wherein a developing liquid is used to transport the analyte and the labeled binding reagent to the second location.

38. A device for detecting an analyte in a sample, comprising
a naturally hydrophilic membrane comprising a first location and a second location,
said first location comprises a dried first fluorescent or luminescent particle labeled binding reagent capable of binding to an analyte, and a dried second binding reagent capable of binding to an analyte, said second binding reagent further comprising a third binding reagent, said analyte, if present in a sample to be tested, forms a sandwich complex comprising said first labeled binding reagent, said analyte and said second binding reagent;
said second location, downstream from said first location, comprises an immobilized fourth binding reagent capable of binding to said third binding reagent;
a liquid is used to transport said analyte, said first labeled binding reagent and said second binding reagent to said second location whereby said sandwich complex is immobilized at said second location via binding between said third binding reagent and said immobilized fourth binding reagent,
and whereby the presence, absence and/or amount of said analyte in said sample is determined by assessing fluorescence or luminescence comprised in said sandwich complex at said second location.

39. A method for detecting an analyte in a sample, comprising
a) contacting a sample with the device of claim 38, wherein the sample is applied to a site of the membrane upstream of the second site;
b) transporting an analyte, if present in said sample, the dried first fluorescent or luminescent particle labeled binding reagent capable of binding to an analyte, and the dried second binding reagent capable of binding to an analyte to the second location, where a sandwich complex comprising said first labeled binding reagent, said analyte and said second binding reagent is immobilized at said second location via binding between said third binding reagent and said immobilized fourth binding reagent; and
c) determining the presence, absence and/or amount of said analyte in said sample by assessing fluorescence or luminescence comprised in said sandwich complex at said second location.

40. A device for detecting an analyte in a sample, comprising
a) a container containing a liquid or dried first fluorescent or luminescent particle labeled binding reagent capable of binding to an analyte, if present in a sample to be tested, to form a first complex comprising said first labeled binding reagent and said analyte; and
b) a naturally hydrophilic membrane comprising a test location comprising an immobilized second binding reagent capable of binding to said first complex, if present, to form a second complex comprising said first labeled binding reagent, said analyte and said immobilized second binding reagent,
wherein a liquid is used to laterally transport said analyte and said first labeled binding reagent on said membrane to said test location to form said second complex,
and whereby the presence, absence and/or amount of said analyte in said sample is determined by assessing fluorescence or luminescence comprised in said second complex at said test location.

41. A device for detecting an analyte in a sample, comprising:
a naturally hydrophilic membrane comprising a first location and a second location,
said first location comprises a dried fluorescent or luminescent particle labeled binding reagent capable of binding to an analyte, if present in a sample to be tested;
said second location, downstream from said first location, comprises an immobilized substance capable of binding to said labeled binding reagent;
a liquid is used to transport said analyte and said labeled binding reagent to said second location where said analyte and said immobilized substance compete for binding to said labeled binding reagent,
and whereby the presence, absence and/or amount of said analyte in said sample is determined by assessing fluorescence or luminescence comprised in a complex comprising said immobilized substance and said labeled binding reagent at said second location.

42. The device of claim 41, wherein the labeled binding reagent binds specifically to the analyte.

43. The device of claim 41, wherein the labeled binding reagent does not bind specifically to the analyte, and a scavenger substance is used to improve detection specificity.

44. The device of claim 41, wherein the labeled binding reagent is an antibody to the analyte.

45. The device of claim 41, wherein the immobilized substance comprises an analyte.

46. A method for detecting an analyte in a sample, comprising
a) contacting a sample with the device of claim 41, wherein the sample is applied to a site of the membrane upstream of the second site;
b) transporting an analyte, if present in said sample, and the dried first fluorescent or luminescent particle labeled binding reagent capable of binding to an analyte by a liquid to the second location, where said analyte and said immobilized substance compete for binding to said labeled binding reagent; and
c) determining the presence, absence and/or amount of said analyte in said sample by assessing fluorescence or luminescence comprised in a complex comprising said labeled binding reagent and said immobilized substance at said second location.

47. A device for detecting an analyte in a sample, comprising
a naturally hydrophilic membrane comprising a first location and a second location,
said first location comprises a dried fluorescent or luminescent particle labeled substance;
said second location, downstream from said first location, comprises an immobilized binding reagent capable of binding to said labeled substance and an analyte, if present in a sample to be tested;
a liquid is used to transport said analyte and said labeled substance to said second location where said analyte and said labeled substance compete for binding to said immobilized binding reagent,
and whereby the presence, absence and/or amount of said analyte in said sample is determined by assessing fluorescence or luminescence comprised in a complex comprising said immobilized binding reagent and said labeled substance at said second location.

48. The device of claim 47, wherein the immobilized binding reagent binds specifically to the analyte.

49. The device of claim 47, wherein the immobilized binding reagent does not bind specifically to the analyte, and a scavenger substance is used to improve detection specificity.

50. The device of claim 47, wherein the immobilized binding reagent is an antibody to the analyte.

51. The device of claim 47, wherein the labeled substance comprises an analyte.

52. A method for detecting an analyte in a sample, comprising
a) contacting a sample with the device of claim 47, wherein the sample is applied to a site of the membrane upstream of the second site;
b) transporting an analyte, if present in said sample, and the dried fluorescent or luminescent particle labeled substance by a liquid to the second location, where said analyte and said labeled substance compete for binding to said immobilized binding reagent; and
c) determining the presence, absence and/or amount of said analyte in said sample by assessing fluorescence or luminescence comprised in a complex comprising said labeled substance and said immobilized binding reagent at said second location.

53. A device for detecting an analyte in a sample, which device comprises:
a naturally hydrophilic membrane comprising a first location and a second location,
said first location comprises a dried first fluorescent or luminescent particle labeled binding reagent capable of binding to an analyte, and a dried substance further comprising a second binding reagent, said analyte, if present in a sample to be tested, and said substance compete for binding to said first labeled binding reagent;
said second location, downstream from said first location, comprises an immobilized third binding reagent capable of binding to said second binding reagent;
a liquid is used to transport said analyte, said first labeled binding reagent and said substance to said second location whereby a complex comprising said first labeled binding reagent and said substance is immobilized at said second location via binding between said second binding reagent and said immobilized third binding reagent,
and whereby the presence, absence and/or amount of said analyte in said sample is determined by assessing fluorescence or luminescence comprised in said complex comprising said first labeled binding reagent and said substance at said second location.

54. A method for detecting an analyte in a sample, comprising
a) contacting a sample with the device of claim 53, wherein the sample is applied to a site of the membrane upstream of the second site;
b) transporting an analyte, if present in said sample, the first fluorescent or luminescent particle labeled binding reagent capable of binding to said analyte, and the dried substance further comprising a second binding reagent to the second location, where said analyte and said substance compete for binding to said first labeled binding reagent and a complex comprising said first labeled binding reagent and said substance is immobilized at said second location via binding between said second binding reagent and said immobilized third binding reagent; and
c) determining the presence, absence and/or amount of said analyte in said sample by assessing fluorescence or luminescence comprised in said complex comprising said first labeled binding reagent and said substance at said second location.

55. A device for detecting an analyte in a sample, comprising
a naturally hydrophilic membrane comprising a first location and a second location,
said first location comprises a dried fluorescent or luminescent particle labeled substance, and a dried first binding reagent capable of binding to an analyte, said first binding reagent further comprising a second binding reagent, said analyte, if present in a sample to be tested, and said labeled substance compete for binding to said first binding reagent;
said second location, downstream from said first location, comprises an immobilized third binding reagent capable of binding to said second binding reagent;
a liquid is used to transport said analyte, said labeled substance and said first binding reagent to said second location whereby a complex comprising said labeled substance and said first binding reagent is immobilized at said second location via binding between said second binding reagent and said immobilized third binding reagent,
and whereby the presence, absence and/or amount of said analyte in said sample is determined by assessing fluorescence or luminescence comprised in said complex comprising said labeled substance and said first binding reagent at said second location.

56. A method for detecting an analyte in a sample, comprising
a) contacting a sample with the device of claim 55, wherein the sample is applied to a site of the membrane upstream of the second site;
b) transporting an analyte, if present in said sample, the dried fluorescent or luminescent particle labeled substance, and the dried first binding reagent capable of binding to said analyte, said first binding reagent further comprising a second binding reagent, to the second location, where said analyte and said labeled substance compete for binding to said first binding reagent, and a complex comprising said labeled substance and said first binding reagent is immobilized at said second location via binding between said second binding reagent and said immobilized third binding reagent; and
c) determining presence, absence and/or amount of said analyte in said sample by assessing fluorescence or luminescence comprised in said complex comprising said labeled substance and said first binding reagent at said second location.

57. A device for detecting an analyte in a sample, comprising
a) a container containing a liquid or dried fluorescent or luminescent particle labeled binding reagent capable of binding to an analyte, if present in a sample to be tested; and
b) a naturally hydrophilic membrane comprising a test location comprising an immobilized substance capable of binding to said labeled binding reagent;
wherein a liquid is used to laterally transport said analyte and said labeled binding reagent on said membrane to said test location where said analyte and said immobilized substance compete for binding to said labeled binding reagent,
and whereby presence, absence and/or amount of said analyte in said sample is determined by assessing fluorescence or luminescence comprised in a complex comprising said immobilized substance and said labeled binding reagent at said test location.

58. A device for detecting an analyte in a sample, which device comprises:
a) a container containing a liquid or dried fluorescent or luminescent particle labeled substance; and
b) a naturally hydrophilic membrane comprising a test location comprising an immobilized binding reagent capable of binding to said labeled substance and an analyte, if present in a sample to be tested;
wherein a liquid is used to laterally transport said analyte and said labeled substance on said membrane to said test location where said analyte and said labeled substance compete for binding to said immobilized binding reagent,
and whereby presence, absence and/or amount of said analyte in said sample is determined by assessing fluorescence or luminescence comprised in a complex comprising said immobilized binding reagent and said labeled substance at said test location.

59. A lateral flow device for detection of an analyte in a sample fluid; wherein said analyte comprises at least two distinct epitopes, said lateral flow device comprising
a single membrane;
mobile fluorescent or luminescent detection moieties, present on a storage zone of said membrane, capable of binding to a first epitope on said analyte;
capture moieties, capable of binding to a second epitope on said analyte, immobilized on a detection zone in said membrane;
a sample application zone on said membrane; and a user controlled reservoir containing a transport liquid;
wherein after application of the sample, user controlled manipulation of the reservoir releases a transport liquid onto the membrane, transporting the analyte to the fluorescent or luminescent detection moieties, which bind to a first epitope on the analyte, and then transports the analyte and mobile fluorescent or luminescent detection moieties to the detection zone on said membrane, where capture moieties bind to a second epitope on said analyte, producing a detectable signal.

60. The device of embodiment 59, in which the detection moieties and detection moieties are proteins selected from the group consisting of antibodies, receptors, and lectins.

61. A lateral flow device for detection of an analyte in a sample fluid; wherein said analyte comprises at least two distinct epitopes, said lateral flow device comprising
mobile detection moieties present on a storage zone on said membrane, which generate a detectable signal, capable of binding to a first epitope on said analyte;
capture moieties, capable of binding to a second epitope on said analyte, immobilized on a detection zone in said membrane;
a sample application zone on said membrane;
and sample detection electrodes associated with said sample application zone on said membrane;
wherein application of sample to said application zone produces a change in an electrical property across said sample detection electrodes, enabling the time of sample application to be automatically assessed by an instrument that monitors the electrical state of said sample detection electrodes.

62. A lateral flow device for detection of an analyte in a sample fluid; wherein said analyte comprises at least two distinct epitopes, said lateral flow device comprising
mobile detection moieties present on a storage zone on said membrane, which generate a detectable signal, and are capable of binding to a first epitope on said analyte;
capture moieties, capable of binding to a second epitope on said analyte, immobilized on a detection zone in said membrane;
a sample application zone on said membrane; and
fluid transport detection electrodes associated with said detection zone on said membrane;
wherein fluid transport occurring after application of sample to said application zone produces a change in an electrical property across said detection electrodes, enabling the time of sample transport past the detection zone region to be automatically assessed by an instrument that monitors the electrical state of said detection zone electrodes.

63. An instrument for reading a lateral flow immunoassay device, said instrument comprising
a fluorescence or luminescence detector capable of reading a detection zone on said immunoassay device;
electrodes capable of interfacing with electrodes on said lateral flow immunoassay device;
and computation means,
wherein travel of fluid in the lateral flow immunoassay device causes a change in electrical property across the electrodes on said lateral flow immunoassay device, said change in electrical property is communicated to the instrument via the instrument's interface electrodes, and the information is transmitted to the instrument's computational means.

64. A validation method for a lateral flow device for detection of an analyte in a sample fluid, wherein said validation method comprises
an analyte detection particle and a control detection particle said analyte detection particle containing means to specifically bind to a first epitope on the analyte;
said analyte detection particle emitting a first detectable signal;
said control detection particle containing means to specifically bind to a region on a control non-analyte molecule;
said control detection particle emitting a second detectable signal, which may be distinguished from the first detectable signal;
a detection zone on said lateral flow device containing means to specifically bind to a second epitope on the analyte, said means being immobilized to avoid migration away from said detection zone;
said detection zone further containing means to specifically bind to a region on said control non-analyte molecule that is different from the region bound by the control detection particle, said means being immobilized to avoid migration away from said detection zone; said analyte binding means and said control binding means being intermixed in the same region of said detection zone;
wherein validation of said lateral flow device is achieved by monitoring the binding or non binding of the control particle to the control non-analyte molecule by means of the second detectable signal emitted by the control particle.

65. A method for extending the analyte detection dynamic range of a lateral flow device for detection of an analyte in a sample fluid, comprising
a high affinity analyte detection particle and a high affinity detection particle said high affinity analyte detection particle containing means to specifically bind to a first epitope on the analyte with a strong binding force;
said high affinity analyte detection particle emitting a first detectable signal; and
a low affinity analyte detection particle;
said low affinity analyte detection particle containing means to specifically bind to an epitope on the analyte with a weak binding force;
said low affinity detection particle emitting a second detectable signal, which may be distinguished from the first detectable signal;
a detection zone on said lateral flow device containing means to specifically bind to a second epitope on the analyte, said means being immobilized to avoid migration away from said detection zone;
wherein low analyte concentrations are detected by monitoring the binding or non binding of the high affinity detection particle to the detection zone by means of the first detectable signal, and high an alyte concentrations are detected by monitoring the binding or non-binding of the low affinity detection particle to the detection zone by means of the second detectable signal.

66. A method to modify the surface properties of a particle to render the particle, when suspended in an aqueous carrier solvent, capable of migrating within the matrix of a bibulous membrane without adverse interaction with other particles or with the bibulous membrane matrix, said method comprising:
covalently modifying the surface of the particle with one or more ligands, said ligands being capable of binding to hydrophilic molecules by a covalent or non-covalent bond;
said hydrophilic molecules being soluble in said aqueous carrier solvent;
wherein said hydrophilic molecules protect the particle from adverse interactions with other particles or with the bibulous membrane matrix.

67. The method of embodiment 66 in which said adverse interaction comprises particle aggregation or particle sticking to the bibulous membrane matrix.

68. The method of embodiment 66 in which said ligand is selected from the group consisting of strepavidin or avidin, and said hydrophilic molecule is selected from the group consisting of biotinated bovine serum albumin, or other biotinated hydrophilic protein, and the binding reaction is quenched with excess biotin.

69. The method of embodiment 66, in which the ligands are hydrophilic, and are capable of protecting the particle from adverse interactions with other particles or with the bibulous membrane matrix without the need of binding to additional hydrophilic molecules.
